(19) United States
(12) Patent Application Publication
(10) Pub. No.: US 2019/0029758 A1
(43) Pub. Date: Jan. 31, 2019

(54) MRI TARGETING SET WITH IMPROVED TARGETING SLEEVE

(71) Applicant: Devicor Medical Products, Inc., Cincinnati, OH (US)

(72) Inventors: Robert M. Householder, Cincinnati, OH (US); Rachel Yoon Choung, Cincinnati, OH (US); John Kevin Bruce, Burlington, KY (US)

(21) Appl. No.: 15/984,715

(22) Filed: May 21, 2018

Related U.S. Application Data
(60) Provisional application No. 62/509,496, filed on May 22, 2017.

(51) Int. Cl. A61B 34/20; A61B 10/02

(52) U.S. Cl. CPC A61B 34/20; A61B 2090/374; A61B 2034/2051; A61B 10/0275

(57) ABSTRACT

A targeting set for use with positioning a biopsy needle relative to a patient. The targeting set includes an obturator, a side notch, and a neck. The obturator includes an elongate shaft extending between a distal tip and a hub. The side notch is defined by the elongate shaft and is configured to receive tissue for visualization under MRI. The neck is defined by the elongate shaft and disposed proximate of the side notch. The neck has a tapered portion tapering radially inwardly towards the distal tip.

### PRIORITY

This application claims priority to U.S. Provisional Patent App. No. 62/509,496 entitled “MRI Targeting Set with Improved Targeting Sleeve,” filed May 22, 2017, the disclosure of which is incorporated by reference herein.

### BACKGROUND

Biopsy samples have been obtained in a variety of ways in various medical procedures using a variety of devices. Biopsy devices may be used under stereotactic guidance, ultrasound guidance, MRI guidance, PEM guidance, BSGI guidance, or otherwise. For instance, some biopsy devices may be fully operable by a user using a single hand, and with a single insertion, to capture one or more biopsy samples from a patient. In addition, some biopsy devices may be tethered to a vacuum module and/or control module, such as for communication of fluids (e.g., pressurized air, saline, atmospheric air, vacuum, etc.), for communication of power, and/or for communication of commands and the like. Other biopsy devices may be fully or at least partially operable without being tethered or otherwise connected with another device.

Merely exemplary biopsy devices and biopsy system components are disclosed in U.S. Pat. No. 5,526,822, entitled “Method and Apparatus for Automated Biopsy and Collection of Soft Tissue,” issued Jun. 18, 1996; U.S. Pat. No. 6,017,316, entitled “Vacuum Control System and Method for Automated Biopsy Device,” issued Jan. 25, 2000; U.S. Pat. No. 6,086,544, entitled “Control Apparatus for an Automated Surgical Biopsy Device,” issued Jul. 11, 2000; U.S. Pat. No. 6,432,065, entitled “Method for Using a Surgical Biopsy System with Remote Control for Selecting an Operational Mode,” issued Aug. 13, 2002; U.S. Pat. No. 7,442,171, entitled “Remote Thumbwheel for a Surgical Biopsy Device,” issued Oct. 8, 2008; U.S. Pat. No. 7,938,786, entitled “Vacuum Timing Algorithm for Biopsy Device,” issued May 10, 2011; U.S. Pat. No. 8,083,687, entitled “Tissue Biopsy Device with Rotatably Linked Thumbwheel and Tissue Sample Holder,” issued Dec. 21, 2011; U.S. Pat. No. 8,206,316, entitled “Tetherless Biopsy Device with Reusable Portion,” issued Jun. 26, 2012; U.S. Pat. No. 8,241,226, entitled “Biopsy Device with Rotatable Tissue Sample Holder,” issued Aug. 14, 2012; U.S. Pat. No. 8,702,623, entitled “Biopsy Device with Discrete Tissue Chambers,” issued Apr. 22, 2014; U.S. Pat. No. 8,764,680, entitled “Handheld Biopsy Device with Needle Firing,” issued Jul. 1, 2014; U.S. Pat. No. 8,938,285, entitled “Access Chamber and Markers for Biopsy Device,” issued Jan. 20, 2015; U.S. Pat. No. 8,858,465, entitled “Biopsy Device with Motorized Needle Firing,” issued Oct. 14, 2014; and U.S. Pat. No. 9,326,755, entitled “Biopsy Device Tissue Sample Holder with Bulk Chamber and Pathology Chamber,” issued May 3, 2016. The disclosure of each of the above-cited U.S. Patents is incorporated by reference herein.

Additional exemplary biopsy devices and biopsy system components are disclosed in U.S. Pat. Pub. No. 2006/0074345, entitled “Biopsy Apparatus and Method,” published Apr. 6, 2006, now abandoned; U.S. Pat. Pub. No. 2009/0131821, entitled “Graphical User Interface For Biopsy System Control Module,” published May 21, 2009, now abandoned; U.S. Pat. Pub. No. 2010/0152610, entitled “Hand Actuated Tetherless Biopsy Device with Pistol Grip,” published Jun. 17, 2010, now abandoned; U.S. Pat. Pub. No. 2010/0160819, entitled “Biopsy Device with Central Thumbwheel,” published Jun. 24, 2010, now abandoned; and U.S. Pat. Pub. No. 2013/0324882, entitled “Control for Biopsy Device,” published Dec. 5, 2013, now abandoned. The disclosure of each of the above-cited U.S. Patent Application Publications, U.S. Non-Provisional Patent Applications, and U.S. Provisional Patent Applications is incorporated by reference herein.

In U.S. Pat. No. 7,831,290, entitled “MRI Biopsy Device Localization Fixture” issued on Nov. 9, 2010, the disclosure of which is incorporated by reference herein, a localization mechanism, or fixture, is described that is used in conjunction with a breast coil for breast compression and for guiding a core biopsy instrument during prone biopsy procedures in both open and closed Magnetic Resonance Imaging (MRI) machines. The localization fixture includes a three-dimensional Cartesian positionable guide for supporting and orienting an MRI-compatible biopsy instrument, and, in particular, a cannula/sleeve to a biopsy site of suspicious tissues or lesions. Another merely illustrative localization mechanism used for guiding a core biopsy instrument is disclosed in U.S. Pat. No. 7,507,210, entitled “Biopsy Cannula Adjustable Depth Stop,” issued Mar. 24, 2009, the disclosure of which is incorporated by reference herein. The localization mechanism includes a grid plate configured to removably receive a guide cube capable of supporting and orienting an MRI-compatible biopsy instrument. For instance, a combination of an obturator and targeting cannula/sleeve may be introduced through a breast to a biopsy site via the guide cube, with proper positioning confirmed using MRI imaging. The obturator may then be removed and the needle of a biopsy device may then be inserted through the targeting cannula/sleeve to reach the targeted lesion.

While several systems and methods have been made and used for obtaining a biopsy sample, it is believed that no one prior to the inventor has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements. In the drawings some components or portions of components are shown in phantom as depicted by broken lines.

FIG. 1 depicts a perspective view of a biopsy system including a control module remotely coupled to a biopsy device, and including a localization fixture with a lateral grid plate used in conjunction with a rotatable cube to position an obturator or a probe of the biopsy device to a desired insertion depth as set by a ring stop;

FIG. 2 depicts a perspective view of a breast coil receiving the localization fixture of FIG. 1;

FIG. 3 depicts a perspective view of the biopsy device inserted through the rotatable cube within the cube plate of the localization fixture attached to the breast coil of FIG. 2;

FIG. 4 depicts a perspective view of a two-axis rotatable guide cube of the biopsy system of FIG. 1;

FIG. 5 depicts a diagram of nine guide positions achievable by the two-axis rotatable guide cube of FIG. 4;

FIG. 6 depicts a perspective view of a two-axis rotatable guide cube into a lateral grid with the backing of the localization fixture of FIG. 1;

FIG. 7 depicts a perspective view of a obturator and cannula of the biopsy system of FIG. 1;

FIG. 8 depicts a perspective exploded view of the obturator and cannula of FIG. 7;

FIG. 9 depicts a perspective view of the obturator and cannula of FIG. 7 with a depth stop device of FIG. 1 inserted through the guide cube and grid plate of FIG. 6;

FIG. 10 depicts a perspective view of an exemplary alternative targeting set that may be readily used with the biopsy system of FIG. 1;

FIG. 11 depicts a perspective exploded view of the targeting set of FIG. 10;

FIG. 12 depicts a perspective view of an obturator of the targeting set of FIG. 10;

FIG. 13 depicts a partial perspective view of a cannula of the targeting set of FIG. 10;

FIG. 14 depicts a partial front cross-sectional view of the cannula of FIG. 13;

FIG. 15A depicts a partial perspective view of the obturator of FIG. 12 inserted into the cannula of FIG. 13, with a neck portion positioned within the cannula and a pair of sleeves deformed outwardly;

FIG. 15B depicts another partial perspective view of the obturator of FIG. 12 inserted in the cannula of FIG. 13, with the neck portion of the obturator extended beyond the cannula;

FIG. 16A depicts a partial cross-sectional view of the obturator of FIG. 12 inserted in the cannula of FIG. 13, with the neck portion positioned within the cannula, the pair of sleeves deformed outwardly and the pair of slots in the expanded state, with the cross-section taken along line 16A-16A on FIG. 15A;

FIG. 16B depicts another partial cross-sectional view of the obturator of FIG. 12 inserted in the cannula of FIG. 13, with the neck portion extended beyond the cannula and the pair of sleeves positioned at a proximal end of a ramp, with the cross-section taken along line 16B-16B of FIG. 15B;

FIG. 17 depicts a partial perspective view of an exemplary alternative cannula, with the cannula including a pair of sleeves and a pair of slots, with the pair of slots positioned along a left and right wall of the cannula;

FIG. 18 depicts a partial perspective view of another exemplary alternative cannula, with the cannula including a pair of sleeves and a pair of slots, with the pair of slots having an irregular shape and positioned along a left and right wall of the cannula;

FIG. 19 depicts a partial perspective view of still another exemplary alternative cannula, with the cannula including a pair of sleeves and a longitudinal slit, with the longitudinal slit positioned along a top wall of the cannula;

FIG. 20A depicts a partial perspective view of the cannula of FIG. 19, with the longitudinal slit in an unexpanded state; and

FIG. 20B depicts a partial perspective view of the cannula of FIG. 19, with the longitudinal slit in an expanded state and the pair of sleeves deformed outwardly.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

### I. Overview of Exemplary MRI Biopsy Control Module

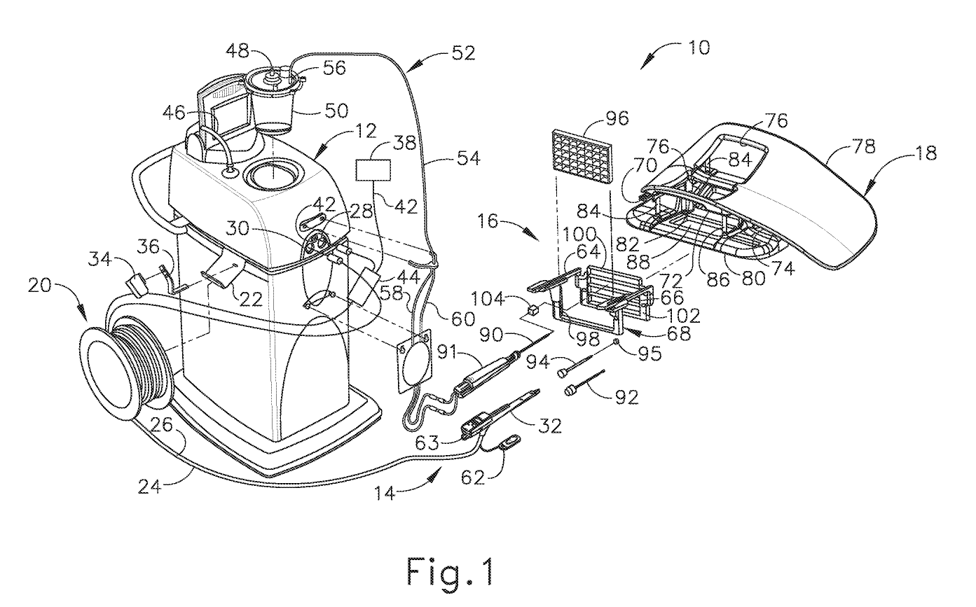

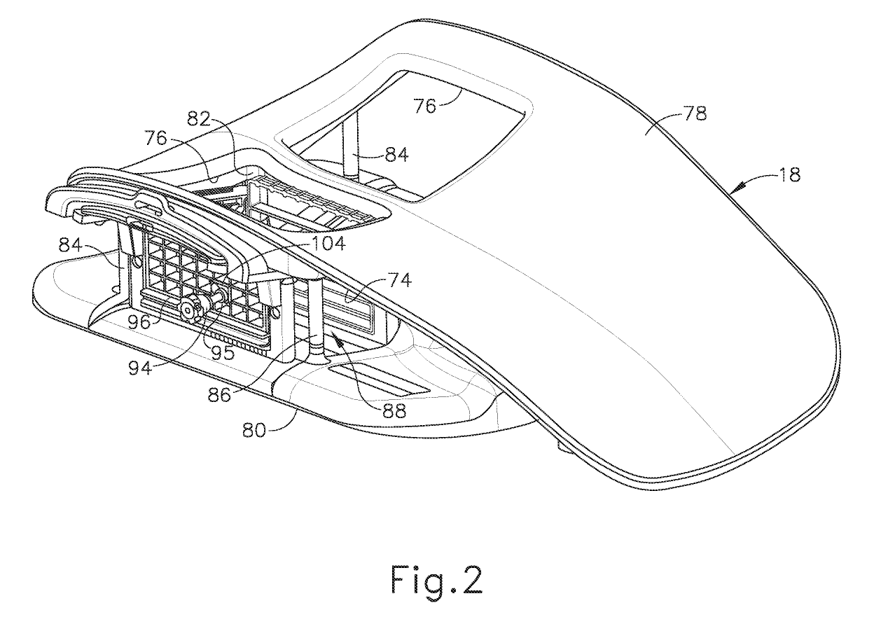

In FIGS. 1-3, an MRI compatible biopsy system (10) has control module (12) that may be placed outside of a shielded room containing an MRI machine (not shown) or at least spaced away to mitigate detrimental interaction with its strong magnetic field and/or sensitive radio frequency (RF) signal detection antennas. As described in U.S. Pat. No. 6,752,768, which is hereby incorporated by reference in its entirety, a range of preprogrammed functionality may be incorporated into control module (12) to assist in taking tissue samples. Control module (12) controls and powers biopsy device (14) that is used with localization assembly (15). Biopsy device (14) is positioned and guided by localization fixture (16) attached to breast coil (18) that may be placed upon a gantry (not shown) of a MRI or other imaging machine.

In the present example, control module (12) is mechanically, electrically, and pneumatically coupled to biopsy device (14) so that components may be segregated that need to be spaced away from the strong magnetic field and the sensitive RF receiving components of a MRI machine. Cable management spool (20) is placed upon cable management attachment saddle (22) that projects from a side of control module (12). Wound upon cable management spool (20) is paired electrical cable (24) and mechanical cable (26) for communicating control signals and cutter rotation/advancement motions respectively. In particular, electrical and mechanical cables (24, 26) each have one end connected to respective electrical and mechanical ports (28, 30) in control module (12) and another end connected to holster portion (32) of biopsy device (14). Docking cup (34), which may hold holster portion (32) when not in use, is hooked to control module (12) by docking station mounting bracket (36). It should be understood that such components described above as being associated with control module (12) are merely optional.

Interface lock box (38) mounted to a wall provides tether (40) to lockout port (42) on control module (12). Tether (40) is uniquely terminated and of short length to preclude inadvertent positioning of control module (12) too close to a MRI machine or other machine. In-line enclosure (44) may register tether (40), electrical cable (24) and mechanical cable (26) to their respective ports (42, 28, 30) on control module (12).

Vacuum assist is provided by first vacuum line (46) that connects between control module (12) and outlet port (48) of vacuum canister (50) that catches liquid and solid debris. Tubing kit (52) completes the pneumatic communication between control module (12) and biopsy device (14). In particular, second vacuum line (54) is connected to inlet port (56) of vacuum canister (50). Second vacuum line (54) divides into two vacuum lines (58, 60) that are attached to biopsy device (14). With biopsy device (14) installed in holster portion (32), control module (12) performs a functional check. Saline may be manually injected into biopsy device (14) or otherwise introduced to biopsy device (14), such as to serve as a lubricant and to assist in achieving a vacuum seal and/or for other purposes. Control module (12) actuates a cutter mechanism (not shown) in biopsy device (14), monitoring full travel of a cutter in biopsy device (14) in the present example. Binding in mechanical cable (26) or within biopsy device (14) may optionally monitored with reference to motor force exerted to turn mechanical cable (26) and/or an amount of twist in mechanical cable (26) sensed in comparing rotary speed or position at each end of mechanical cable (26).

Remote keypad (62), which is detachable from holster portion (32), communicates via electrical cable (24) to control panel (12) to enhance clinician control of biopsy device (14) in the present example, especially when controls that would otherwise be on biopsy device (14) itself are not readily accessible after insertion into localization fixture (16) and/or placement of control module (12) is inconveniently remote (e.g., 30 feet away). However, as with other components described herein, remote keypad (62) is merely optional, and may be modified, substituted, supplemented, or omitted as desired. In the present example, aft end thumbwheel (63) on holster portion (32) is also readily accessible after insertion to rotate the side from which a tissue sample is to be taken.

Of course, the above-described control module (12) is merely one example. Any other suitable type of control module (12) and associated components may be used. By way of example only, control module (12) may instead be configured and operable in accordance with the teachings of U.S. Pub. No. 2008/0228103, entitled “Vacuum Timing Algorithm for Biopsy Device,” published Sep. 18, 2008, the disclosure of which is incorporated by reference herein. As another merely illustrative example, control module (12) may instead be configured and operable in accordance with the teachings of U.S. Pat. No. 8,328,732, entitled “Control Module Interface for MRI Biopsy Device,” issued Dec. 11, 2012, the disclosure of which is incorporated by reference herein. Alternatively, control module (12) may have any other suitable components, features, configurations, functionalities, operability, etc. Other suitable variations of control module (12) and associated components will be apparent to those of ordinary skill in the art in view of the teachings herein.

### II. Exemplary Localization Assembly

Left and right parallel upper guides (64, 66) of localization framework (68) are laterally adjustably received respectively within left and right parallel upper tracks (70, 72) attached to under side (74) and to each side of a selected breast aperture (76) formed in patient support platform (78) of breast coil (18). Base (80) of breast coil (18) is connected by centerline pillars (82) that are attached to patient support platform (78) between breast apertures (76). Also, a pair of outer vertical support pillars (84, 86) on each side spaced about a respective breast aperture (76) respectively define lateral recess (88) within which localization fixture (16) resides.

It should be appreciated that the patient's breasts hang pendulously respectively into breast apertures (76) within lateral recesses (88) in the present example. For convenience, herein a convention is used for locating a suspicious lesion by Cartesian coordinates within breast tissue referenced to localization fixture (16) and to thereafter selectively position an instrument, such as needle (90) of probe (91) that is engaged to holster portion (32) to form biopsy device (14). Of course, any other type of coordinate system or targeting techniques may be used. To enhance hands-off use of biopsy system (10), especially for repeated re-imaging within the narrow confines of a closed bore MRI machine, biopsy system (10) may also guide obturator (92) encompassed by cannula (94). Depth of insertion is controlled by depth stop device (95) longitudinally positioned on either needle (90) or cannula (94). Alternatively, depth of insertion may be controlled in any other suitable fashion.

This guidance is specifically provided by a lateral fence in the present example, depicted as grid plate (96), which is received within laterally adjustable outer three-sided plate bracket (98) attached below left and right parallel upper guides (64, 66). Similarly, a medial fence with respect to a medial plane of the chest of the patient, depicted as medial plate (100), is received within inner three-sided plate bracket (102) attached below left and right parallel upper guides (64, 66) close to centerline pillars (82) when installed in breast coil (18). To further refine the insertion point of the instrument (e.g., needle (90) of probe (91), obturator/cannula (92, 94), etc.), guide cube (104) may be inserted into grid plate (96).

In the present example, the selected breast is compressed along an inner (medial) side by medial plate (100) and on an outer (lateral) side of the breast by grid plate (96), the latter defining an X-Y plane. The X-axis is vertical (sagittal) with respect to a standing patient and corresponds to a left-to-right axis as viewed by a clinician facing the externally exposed portion of localization fixture (16). Perpendicular to this X-Y plane extending toward the medial side of the breast is the Z-axis, which typically corresponds to the orientation and depth of insertion of needle (90) or obturator/cannula (92, 94) of biopsy device (14). For clarity, the term Z-axis may be used interchangeably with “axis of penetration”, although the latter may or may not be orthogonal to the spatial coordinates used to locate an insertion point on the patient. Versions of localization fixture (16) described herein allow a non-orthogonal axis of penetration to the X-Y axis to a lesion at a convenient or clinically beneficial angle.

It should be understood that the above-described localization assembly (15) is merely one example. Any other suitable type of localization assembly (15) may be used, including but not limited to localization assemblies (15) that use a breast coil (18) and/or localization fixture (16) different from those described above. Other suitable components, features, configurations, functionalities, operability, etc. for a localization assembly (15) will be apparent to those of ordinary skill in the art in view of the teachings herein.

### III. Exemplary Biopsy Device

As shown in FIG. 1, one version of biopsy device (14) may comprise holster portion (32) and probe (91). Exemplary holster portion (32) was discussed previously in the above section addressing control module (12). The following paragraphs will discuss probe (91) and associated components and devices in further detail.

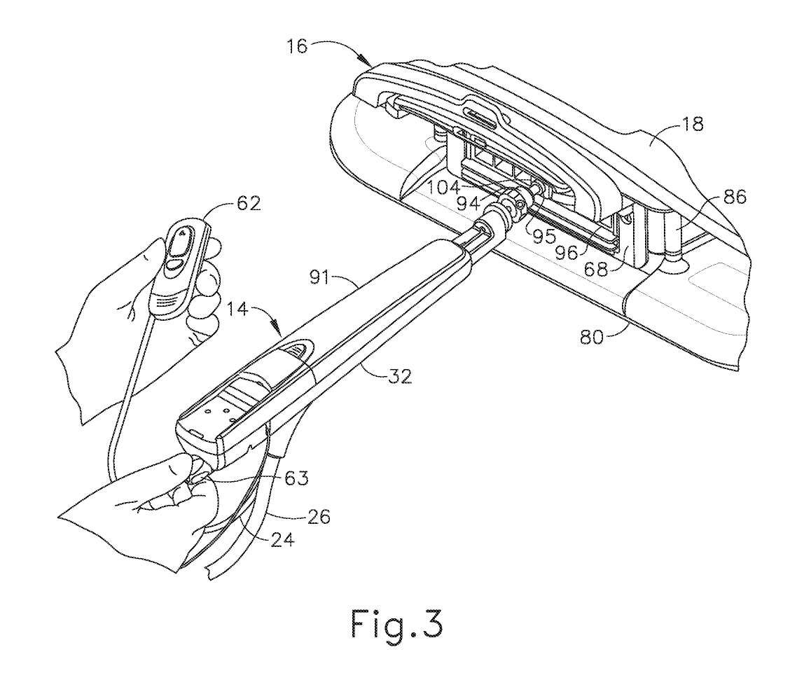

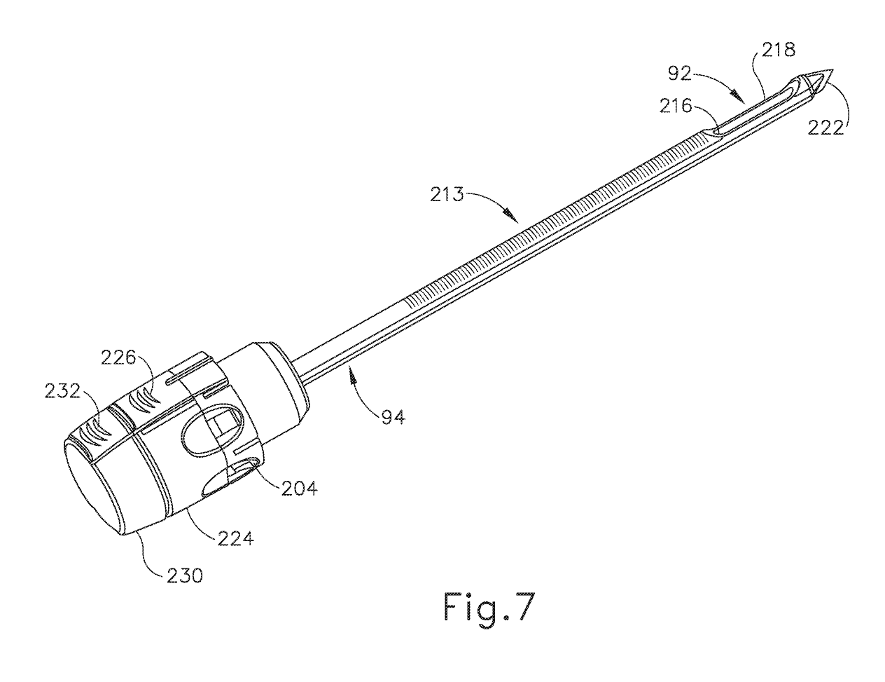

In the present example, a targeting set (89) comprising cannula (94) and obturator (92) is associated with probe (91). In particular, and as shown in FIGS. 7, 8, and 9, obturator (92) is slid into cannula (94) and the combination is guided through guide cube (104) to the biopsy site within the breast tissue. As shown in FIG. 3, obturator (92) is then withdrawn from cannula (94), then needle (90) of probe (91) is inserted in cannula (94), and then biopsy device (14) is operated to acquire one or more tissue samples from the breast via needle (90).

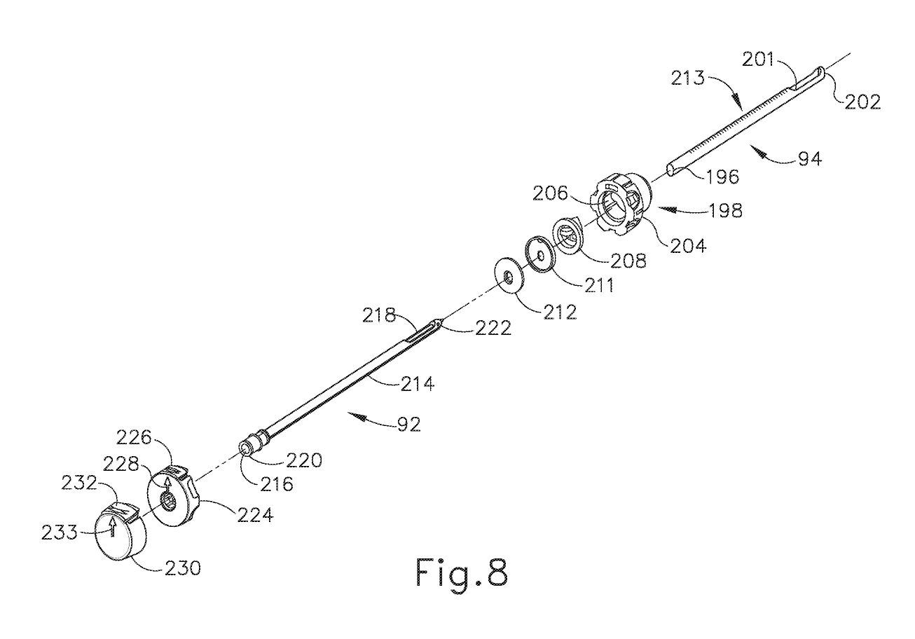

As best seen in FIG. 8, cannula (94) of the present example is proximally attached to cylindrical hub (198) and cannula (94) includes lumen (196) and lateral aperture (201) proximate to open distal end (202). Cylindrical hub (198) has exteriorly presented thumbwheel (204) for rotating lateral aperture (201). Cylindrical hub (198) has interior recess (206) that encompasses duckbill seal (208), wiper seal (211) and seal retainer (212) to provide a fluid seal when lumen (196) is empty and for sealing to inserted obturator (92). Longitudinally spaced measurement indicia (213) along an outer surface of cannula (94) visually, and perhaps physically, provide a means to locate depth stop device (95) of FIG. 1.

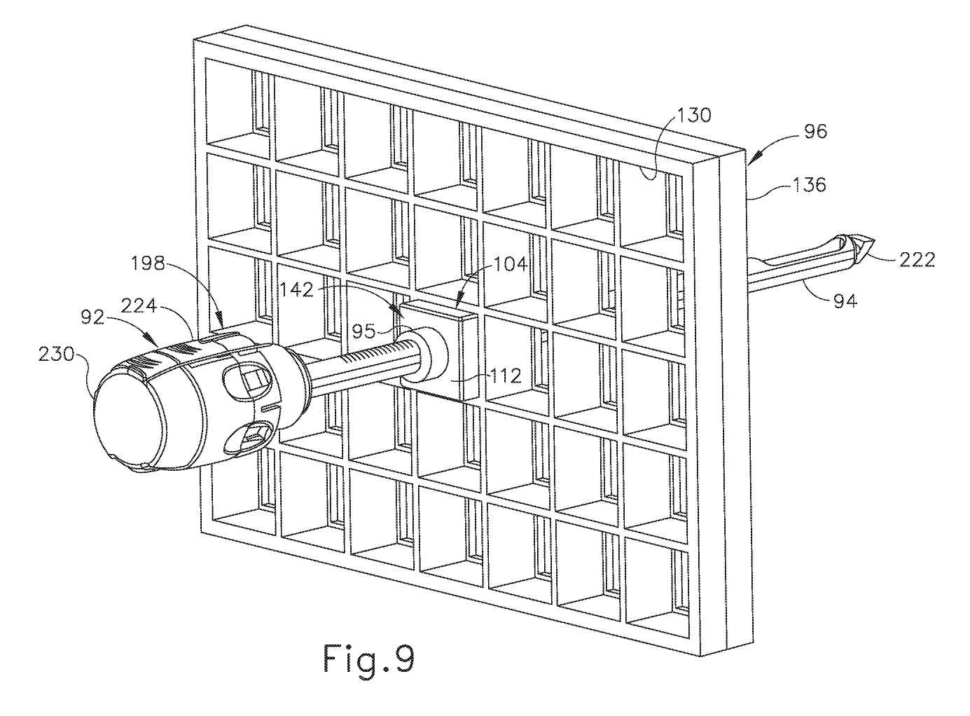

Obturator (92) of the present example incorporates a number of components with corresponding features. For instance, obturator (92) includes a shaft (214) that includes fluid lumen (216) that communicates between imageable side notch/recess (218) and proximal port (220). Shaft (214) is longitudinally sized such that piercing tip (222) extends out of distal end (202) of cannula (94). Obturator thumbwheel cap (224) encompasses proximal port (220) and includes locking feature (226), which includes visible angle indicator (228), that engages cannula thumbwheel (204) to ensure that imageable side notch (218) is registered to lateral aperture (201) in cannula (94). Obturator seal cap (230) may be engaged proximally into obturator thumbwheel cap (224) to close fluid lumen (216). Obturator seal cap (230) of the present example includes locking or locating feature (232) that includes visible angle indicator (233) that corresponds with visible angle indicator (228) on obturator thumbwheel cap (224), which may be fashioned from either a rigid, soft, or elastomeric material. In FIG. 9, guide cube (104) has guided obturator (92) and cannula (94) through grid plate (96).

While obturator (92) of the present example is hollow, it should be understood that obturator (92) may alternatively have a substantially solid interior, such that obturator (92) does not define an interior lumen. In addition, obturator (92) may lack side notch (218) in some versions. Other suitable components, features, configurations, functionalities, operability, etc. for an obturator (92) will be apparent to those of ordinary skill in the art in view of the teachings herein. Likewise, cannula (94) may be varied in a number of ways. For instance, in some other versions, cannula (94) has a closed distal end (202). As another merely illustrative example, cannula (94) may have a closed piercing tip (222) instead of obturator (92) having piercing tip (222). In some such versions, obturator (92) may simply have a blunt distal end; or the distal end of obturator (92) may have any other suitable structures, features, or configurations. Other suitable components, features, configurations, functionalities, operability, etc. for a cannula (94) will be apparent to those of ordinary skill in the art in view of the teachings herein. Furthermore, in some versions, one or both of obturator (92) or cannula (94) may be omitted altogether. For instance, needle (90) of probe (91) may be directly inserted into a guide cube (104), without being inserted into guide cube (104) via cannula (94).

Another component that may be used with probe (91) (or needle (90)) is depth stop device (95). Depth stop device (95) may be of any suitable configuration that is operable to prevent cannula (94) and obturator (92) (or needle (90)) from being inserted further than desired. For instance, depth stop device (95) may be positioned on the exterior of cannula (94) (or needle (90)), and may be configured to restrict the extent to which cannula (94) is inserted into a guide cube. It should be understood that such restriction by depth stop device (95) may further provide a limit on the depth to which the combination of cannula (94) and obturator (92) (or needle (90)) may be inserted into the patient's breast. Furthermore, it should be understood that such restriction may establish the depth within the patient's breast at which biopsy device (14) acquires one or more tissue samples after obturator (92) has been withdrawn from cannula (94) and needle (90) has been inserted in cannula (94). Exemplary depth stop devices (95) that may be used with biopsy system (10) are described in U.S. Pub. No. 2007/0255168, entitled “Grid and Rotatable Cube Guide Localization Fixture for Biopsy Device,” published Nov. 1, 2007, and incorporated by reference herein as mentioned previously.

In the present example, and as noted above, biopsy device (14) includes a needle (90) that may be inserted into cannula (94) after the combination of cannula (94) and obturator (92) has been inserted to a desired location within a patient's breast and after obturator (92) has been removed from cannula (94). Needle (90) of the present example comprises a lateral aperture (not shown) that is configured to substantially align with lateral aperture (201) of cannula (94) when needle (90) is inserted into lumen (196) of cannula (94). Probe (91) of the present example further comprises a rotating and translating cutter (not shown), which is driven by components in holster (32), and which is operable to sever tissue protruding through lateral aperture (201) of cannula (94) and the lateral aperture of needle (90). Severed tissue samples may be retrieved from biopsy device (14) in any suitable fashion.

It should be understood that although biopsy system (10) is discussed above as utilizing disposable probe assembly (91), other suitable probe assemblies and biopsy device assemblies may be utilized. By way of example only, other suitable biopsy devices may be configured and operable in accordance with at least some of the teachings of U.S. Pat. No. 8,206,316, entitled “Tetherless Biopsy Device with Reusable Portion,” issued Jun. 26, 2012, the disclosure of which is incorporated by reference herein; U.S. Pat. No. 8,277,394, entitled “Multi-Button Biopsy Device,” issued Oct. 2, 2012, the disclosure of which is incorporated by reference herein; and/or U.S. Pub. No. 2012/0065542, entitled “Biopsy Device Tissue Sample Holder with Removable Tray,” published Mar. 15, 2012, the disclosure of which is incorporated by reference herein. As yet another merely illustrative example, other suitable biopsy devices may be configured and operable in accordance with at least some of the teachings of U.S. Pat. No. 8,702,623, the disclosure of which is incorporated by reference herein; U.S. Pat. No. 9,486,186, entitled “Biopsy Device with Slide-In Probe,” issued Nov. 8, 2016, the disclosure of which is incorporated by reference herein; U.S. Patent Pub. No. 2013/0324882, entitled “Control for Biopsy Device,” published Dec. 5, 2013, the disclosure of which is incorporated by reference herein; U.S. Patent Pub. No. 2014/0039343, entitled “Biopsy System,” published Feb. 6, 2014, the disclosure of which is incorporated by reference herein; and/or U.S. Patent Pub. No. 2015/0065913, entitled “Tissue Collection Assembly for Biopsy Device,” published Mar. 5, 2015, the disclosure of which is incorporated by reference herein.

Still other suitable forms of biopsy devices that may be used in conjunction with the various alternative components of system (10) as described herein will be apparent to those of ordinary skill in the art.

### IV. Exemplary Guide Cube

In some versions, a guide cube may comprise a body defined by one or more edges and faces. The body may include one or more guide holes or other types of passages that extend between faces of the guide cube and that may be used to guide an instrument such as a biopsy device (14) or a portion of a biopsy device (14) (e.g., needle (90) of biopsy device (14), a combination of cannula (94) and obturator (92), etc.). Guide cubes may be rotatable about one, two, or three axes to position the one or more guide holes or passages of the guide cube into a desired position.

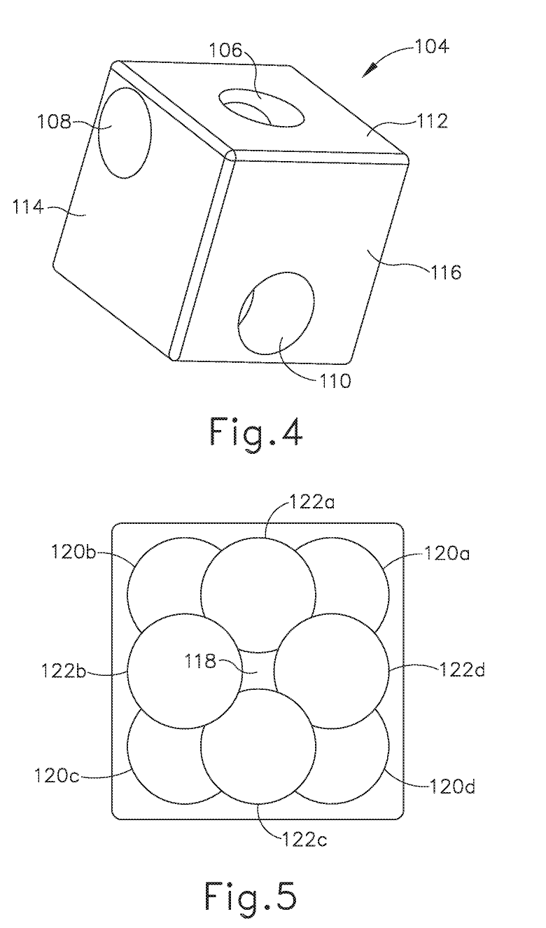

In FIG. 4, guide cube (104) includes a central guide hole (106), a corner guide hole (108), and an off-center guide hole (110) that pass orthogonally to one another between respective opposite pairs of faces (112, 114, 116). By selectively rotating guide cube (104) in two axis, one of pairs of faces (112, 114, 116) may be proximally aligned to an unturned position and then selected proximal face (112, 114, 116) optionally rotated a quarter turn, half turn, or three quarter turn. Thereby, one of nine guide positions (118) (i.e., using central guide hole (106)), (120a-120d) (i.e., corner guide hole (108)), (122a-122d) (i.e., using off-center guide hole (110)) may be proximally exposed as depicted in FIG. 5.

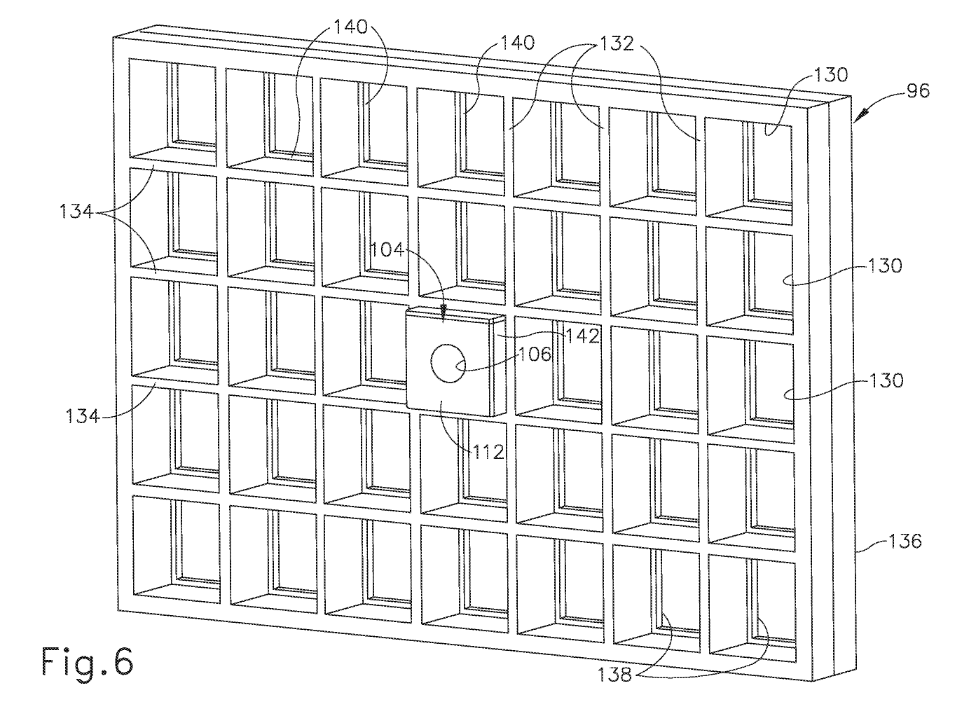

In FIG. 6, two-axis rotatable guide cube (104) is sized for insertion from a proximal side into one of a plurality of square recesses (130) in grid plate (96), which are formed by intersecting vertical bars (132) and horizontal bars (134). Guide cube (104) is prevented from passing through grid plate (96) by backing substrate (136) attached to a front face of grid plate (96). Backing substrate (136) includes respective square opening (138) centered within each square recess (130), forming lip (140) sufficient to capture the front face of guide cube (104), but not so large as to obstruct guide holes (104, 106, 108). The depth of square recesses (130) is less than guide cube (104), thereby exposing a proximal portion (142) of guide cube (104) for seizing and extraction from grid plate (96). It will be appreciated by those of ordinary skill in the art based on the teachings herein that backing substrate (136) of grid plate (96) may be omitted altogether in some versions. In some such versions without backing substrate (136) other features of a guide cube, as will be discussed in more detail below, may be used to securely and removably fit a guide cube within a grid plate. However, such other features may also be used in combination with a grid plate having backing substrate (136), such as grid plate (96), instead of partially or wholly omitting backing substrate (136).

In some other versions, guide cube (104) is replaced with an alternative guide cube or other guide structure that is configured and operable in accordance with at least some of the teachings of U.S. Pat. Pub. No. 2015/0025414, entitled “Biopsy Device Targeting Features,” published Jan. 22, 2015, the disclosure of which is incorporated by reference herein.

### V. Exemplary Alternative Targeting Set

In some instances, it may be beneficial to insert the obturator and targeting cannula/sleeve through a patient's outer skin layer to reach the targeted tissue and/or biopsy site while minimizing the extent of resistance encountered. In a biopsy procedure, as the obturator is inserted into the breast of the patient, the distal end of the cannula/sleeve may cause increased resistance by catching against adjacent skin or tissue. In other words, after overcoming the initial encounter of resistance when puncturing the tip of the obturator through the patient's body, an operator will suddenly encounter a second, and sometimes unanticipated, point of resistance as the distal end of the cannula/sleeve encounters the patient's body. An operator will be required to counteract the subsequent point of resistance by exerting additional force on the obturator and cannula/sleeve combination to overcome the increased resistance and to further penetrate the tissue or skin. This additional point of resistance is not only undesired, it may be unexpected to an operator and detrimental to the patient's health as additional trauma may be inflicted because of the increase in force needed to successfully puncture the obturator into the patient's body.

It may be beneficial to shape the distal end of the obturator in a manner that covers or shields the distal end of the cannula/sleeve to eliminate the additional resistance created as the obturator and cannula/sleeve combination are punctured into the patient's body. In this instance, with the distal end of the cannula/sleeve covered by the obturator, the extent of resistance created by the patient's body may decrease as the occurrence of skin or tissue being caught against the distal end of the cannula/sleeve is minimized.

In cannulas/sleeves such as cannula (94) described above, it may be beneficial to utilize an alternate obturator that has a neck or groove along a portion of the shaft that corresponds to where the distal end of the cannula/sleeve is positioned when the obturator is slidably inserted therein. The neck or groove may be beneficial to cover or shield the distal end of the cannula/sleeve to thereby minimize the resistance encountered by the biopsy device when punctured through the patient's body. It should be understood that the obturator described below may be readily incorporated into any of the various cannulas (94) and biopsy devices (14) described above and in any of the various surgical procedures described in the various references described herein. Other suitable ways in which the below-described obturator may be used will be apparent to those of ordinary skill in the art in view of the teachings herein.

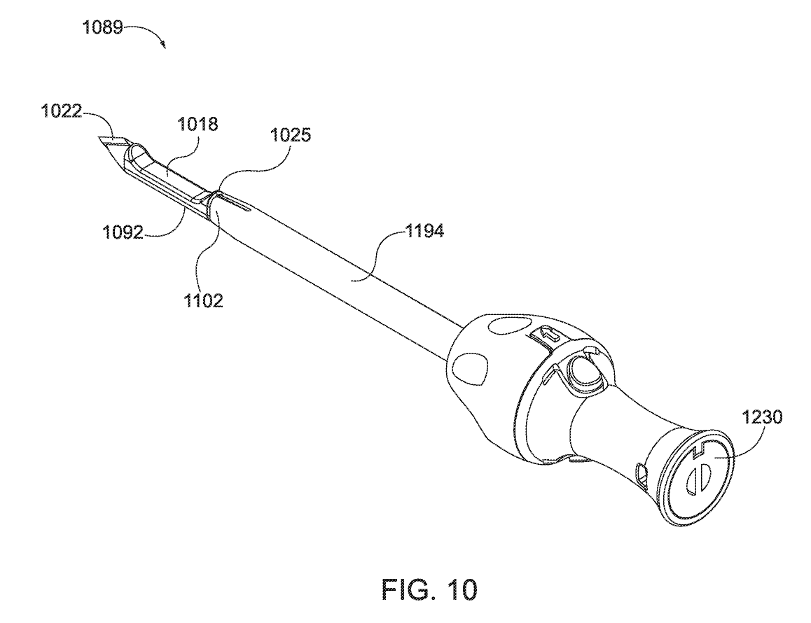

FIG. 10 shows an exemplary alternative targeting set (1089) comprising an obturator (1092) and an exemplary cannula (1194). Obturator (1092) and cannula (1194) except as otherwise described below, may be configured and operable just like obturator (92) and cannula (94), respectively, described above. Obturator (1092) includes a shaft (1014) extending between a piercing tip (1022) and an obturator hub (1230). Shaft (1014) is longitudinally sized such that piercing tip (1022) extends out of distal end (1102) of cannula (1194) when obturator (1092) is slidably received within a lumen (1016) of cannula (1194). Although shaft (1014) in the present example is shown as generally solid in construction, it should be understood that in other examples shaft (1014) can include one or more lumens extending through shaft (1014) such that shaft (1014) can be predominately hollow.

Shaft (1014) of obturator (1092) comprises a generally oval shaped cross-section. As will be understood, the oval-shaped cross-section of shaft (1014) generally corresponds to an oval-shaped cross-section of cannula (1194) such that obturator (1092) and cannula (1194) rotationally interlock when obturator (1092) is disposed within cannula (1194). Obturator (1092) further includes an imageable side notch/recess (1018) along shaft (1014), proximally relative to piercing tip (1022). Side notch (1018) is generally configured to receive tissue therein. As will be understood, receipt of tissue within side notch (1018) generally provides visibility of side notch (1018) under MRI visualization due to differing densities between the portion of obturator (1092) with side notch (1018) and the portion of obturator (1092) without side notch (1018). Although side notch (1018) of the present example is shown as a notch in obturator (1092), it should be understood that in other examples, side notch (1018) can take on a variety of other forms. For instance, in other examples side notch (1018) can be constructed as a side aperture. In addition, in other examples side notch (1018) can be in communication with one or more lumens extending through obturator (1092) to assist with prolapsing tissue into side notch (1018).

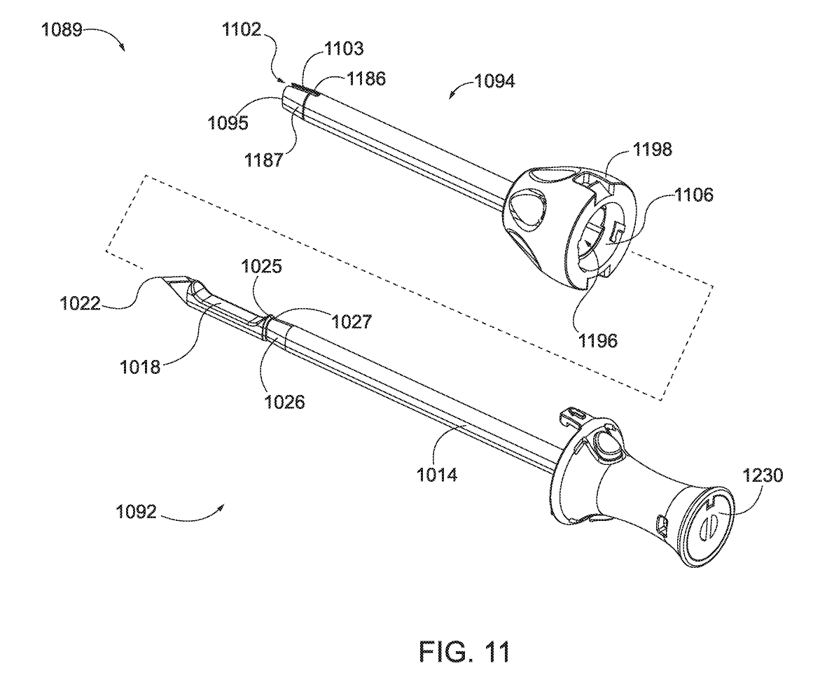

As best seen in FIG. 11, obturator (1092) further includes a neck (1025) and a tapered portion (1026) along shaft (1014). Neck (1025) is positioned along shaft (1014) proximally relative to side notch (1018) and extends outwardly relative to a portion of shaft (1014) along the outer perimeter of shaft (1014). As will be described in greater detail below, neck (1025) is generally configured to receive distal end (1102) of cannula (1194) when obturator (1092) is fully inserted into cannula (1194). In the present example, neck (1025) defines an outward extension from an inwardly oriented tapered portion (1026) defined by the exterior of shaft (1014). This lateral extension defined by neck (1025) is sized to be at least equal to or greater than a thickness (1095) of cannula (1194). Taken from another perspective, the thickness (1095) of cannula is approximately equivalent to one half of the difference between the outer diameter of shaft (1014) minus the smallest inner diameter defined by the taper of neck (1025). In the present example, neck (1025) is integrally formed with obturator (1092). Notwithstanding, it should be understood that in other examples neck (1025) is a separate component selectively attached to obturator (1092).

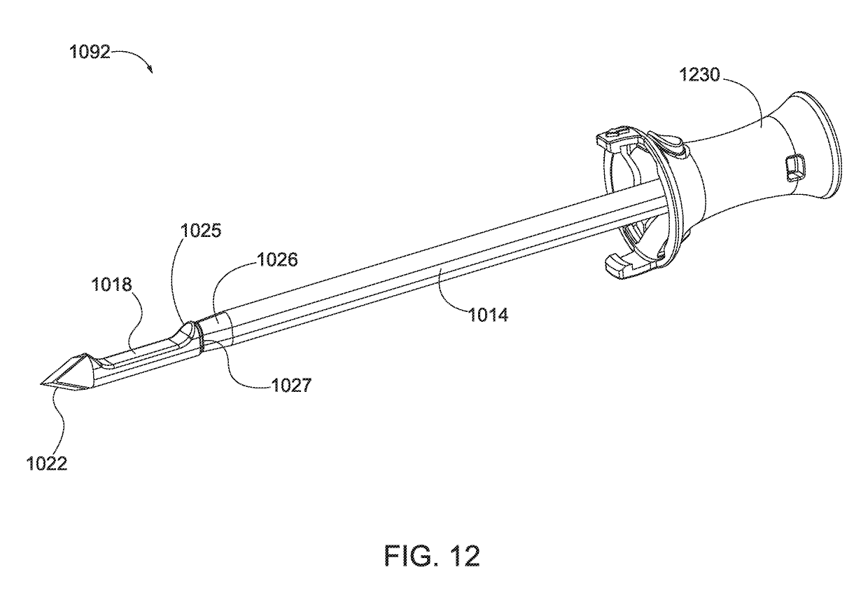

Tapered portion (1026) is disposed adjacently and proximally relative to neck (1025). As best seen in FIG. 12, tapered portion (1026) is extends distally from a portion of shaft (1014) towards neck (1025). At the proximal extent of tapered portion (1026), tapered portion (1026) is generally equivalent in diameter, shape, and/or size to the outer surface of shaft (1014). As tapered portion (1026) extends distally, the size of tapered portion (1026) decreases relative to the size of the outer surface of shaft (1014). In the present example, this decrease in size is at a tapered angle, although such a taper may be varied or omitted in other examples. The size of tapered portion (1026) continues to decrease as tapered portion (1026) extends distally until tapered portion (1026) reaches the interface between tapered portion (1026) and neck (1025).

Obturator (1092) further includes a ramp (1027) positioned between neck (1025) and tapered portion (1026). Ramp (1027) is integrally formed with obturator (1092) and includes a proximal end (1028) and a distal end (1029). Proximal end (1028) is connected to the surface of tapered portion (1026) and distal end (1029) is connected to the extended surface of neck (1025) such that ramp (1027) is configured to provide an intermediate surface extending between and connecting tapered portion (1026) to neck (1025).

As discussed above, it may be beneficial to insert the obturator and targeting cannula/sleeve through a patient's outer skin layer to reach the biopsy site while minimizing the extent of resistance encountered. As the obturator is inserted into the body of a patient, the distal end portion of the cannula/sleeve may cause increased resistance by catching against adjacent skin or tissue. Furthermore, the resistance created by the patient's skin or tissue increases as the cross-sectional area of the cannula/sleeve increases. In other words, when the object being forcibly inserted into the patient's body has a greater cross-sectional area, the skin or tissue of a patient will tighten, thereby increasing the force required to penetrate the tissue. To overcome the increase in resistance, an operator will be required to exert excess force on the obturator and cannula/sleeve combination to overcome the resistance and fully penetrate the tissue or skin. This occurrence is undesired, time consuming, and is potentially detrimental to the patient's wellbeing as additional trauma may be inflicted due to the excess force required to fully puncture the obturator and cannula/sleeve combination into the patient's body.

A cannula/sleeve that is able to form a substantially level surface with the obturator may be desirable to reduce the amount of resistance created by the patient's body as the obturator and cannula combination are introduced through the patient's skin or tissue and towards the target biopsy site. Providing a cannula/sleeve that has a distal end capable of having a narrower profile at the interface point along the shaft of the obturator may be beneficial to create a substantially level surface along the cannula/sleeve and obturator combination. This configuration may help minimize the occurrences of a patient's skin or tissue being caught against the distal end of the cannula/sleeve after the obturator has initially inserted into the patient's body. Additionally, providing a cannula/sleeve that has a tapered profile at the distal end may also be desirable to decrease the cross-sectional area of the sleeve and minimize the occurrence of adjacent tissue or skin snagging against the cannula/sleeve as it is inserted into the patient's body.

In obturators such as obturator (92, 1094) described above, it may be beneficial to utilize an alternate cannula/sleeve that includes a narrowed and/or tapered distal end configured to create a substantially level surface along the external surface of the cannula and obturator combination to thereby reduce the resistance encountered when the obturator and cannula/sleeve combination is inserted into the patient's body. It should be understood that the cannula/sleeve described below may be readily incorporated into any of the various obturators (92, 1092) and biopsy devices (14) described above and in any of the various surgical procedures described in the various references described herein. Other suitable ways in which the below-described cannula/sleeve may be used will be apparent to those of ordinary skill in the art in view of the teachings herein. Except as otherwise described below, cannula (1194) may be configured and operable just like cannula (94) described above.

As best seen in FIG. 11, cannula (1194) is proximally attached to a cylindrical hub (1198) and includes a lumen (1196) and a pair of slots (1103) proximal to an open distal end (1102). Cylindrical hub (1198) includes a recess (1106) that is in communication with lumen (1196) and is configured to receive obturator (1094) therein such that piercing tip (1022) extends distally from open distal end (1102) of cannula (1194). As will be described in greater detail below, recess (1106) of cylindrical hub (1198) is generally configured to receive obturator (1094) to permit at least a portion of obturator (1094) to extend through lumen (1196) and distally through open distal end (1102). As will also be described in greater detail below, slots (1103) are each configured to permit some flexibility of open distal end (1102) to permit cannula (1194) to receive certain features of obturator (1094).

In the present example, cylindrical hub (1198) is selectively couplable to obturator hub (1230) via a dual-latch coupling mechanism. In this dual-latch coupling mechanism, two opposing latches extend from obturator hub (1230) to selectively fasten to corresponding features disposed within cylindrical hub (1198). Although a dual-latch coupling mechanism is described as being used in the present example, it should be understood that in other examples numerous alternative coupling mechanisms may be used. Moreover, in some examples, dual-latch coupling mechanism is configured in accordance with at least some of the teachings of U.S. App. No. 62/509,485, entitled “Method for Securely Attaching and Detaching Obturator to Introducer,” filed on May 22, 2017, the disclosure of which is incorporated by reference herein.

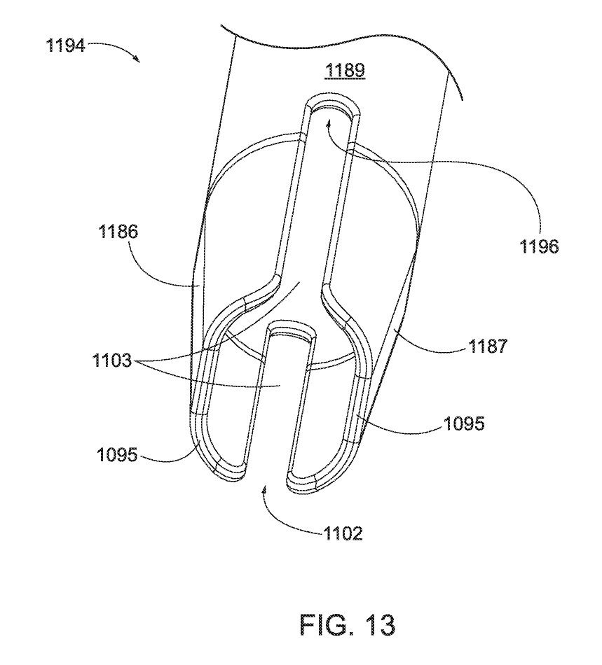

Cannula (1194) is shown in greater detail in FIG. 13. As can be seen, cannula (1194) defines a generally oval-shaped cross-section that corresponds to the oval-shape cross-section of obturator (1094). As described above, this configuration promotes a rotational lock when obturator (1094) is disposed within cannula (1194). Although obturator (1094) and cannula (1194) of the present example are shown and described as including an oval-shaped cross-section, it should be understood that in other examples numerous other shapes may be used. For instance in some examples, obturator (1094) and cannula (1194) may be cylindrical, egg-shaped, or any other suitable shape as will be apparent to those of ordinary skill in the art in view of the teachings herein.

Slots (1103) of cannula (1194) extend proximally from open distal end (1102) of cannula (1194). Slots (1103) are located along a top wall (1189) and a bottom wall (1188) of cannula (1194) such that a left sleeve (1187) and a right sleeve (1186) is formed along a distal portion of cannula (1194) between slots (1103). Slots (1103) are sized and shaped to form an elliptical or oval opening along walls (1188, 1189) to thereby allow sleeves (1186, 1187) to flexibly deflect inwards or outwards relative to lumen (1196) by expanding or contracting slots (1103). Although slots (1103) are depicted as oval openings in FIG. 13, it should be understood that slots (1103) may be sized and shaped to form various suitable openings along the distal end portion of cannula (1194). Although two slots (1103) are depicted, it should be understood that more or less slots (1103) may be included along cannula (1194) as will be apparent to those of ordinary skill in the art.

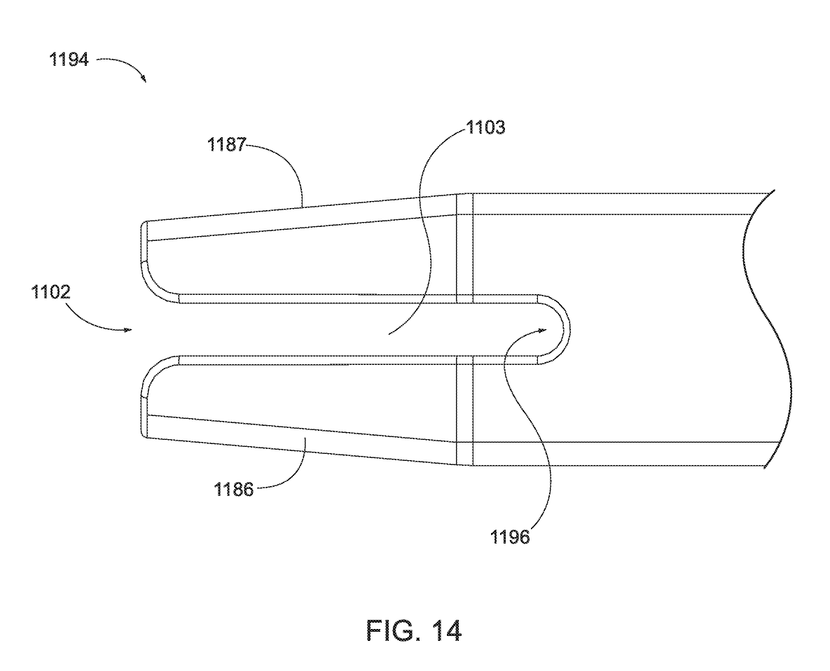

As seen in FIG. 14, each sleeve (1186, 1187) defines a wall thickness that is generally equivalent to the wall thickness defined by wall (1189) of cannula (1194). Thus, wall (1189) of cannula (1194) has a uniform thickness across its entire length in the present example. However, it should be understood that in some examples, wall (1189) includes a non-uniform thickness. For instance, in examples with a non-uniform wall thickness, each sleeve (1186, 1187) is tapered to decrease in thickness as cannula (1194) extends distally. Thus, in examples with a non-uniform wall (1189) thickness, open distal end (1102) of cannula (1194) may have a thin thickness relative to the rest of cannula (1194). This configuration may be desirable to decrease tissue drag at the transition between obturator (1092) and cannula (1194). Of course, it should be understood that in examples where each sleeve (1186, 1187) is tapered, features of obturator (1092) may be correspondingly reconfigured to support the different thickness. For instance, tapered portion (1026) may be increased in size to accommodate each sleeve (1186, 1187). Alternatively, tapered portion (1026) and/or neck (1025) may be eliminated entirely.

Sleeves (1186, 1187) extend along cannula (1194) at an inward angle towards open distal end (1102) such that sleeves (1186, 1187) are configured to deflect into lumen (1196). Cannula (1194) is generally comprised of a stiff, yet flexible material to provide at least some resiliency to sleeves (1186, 1187). Thus, sleeves (1186, 1187) are generally resiliently biased towards the position shown in FIG. 14. However, with obturator (1092) received within lumen (1196) of cannula (1194), sleeves (1186, 1187) are configured to deform outwardly from lumen (1196). As will be understood, this configuration generally promotes passage of neck (1025) of obturator (1092) through open distal end (1102) of cannula (1194), even though neck (1025) has a size or diameter greater than the diameter of lumen (1196) defined by sleeves (1186, 1187). Accordingly, slots (1103) are configured to expand due to the outward force applied onto sleeves (1186, 1187) by neck (1025) being positioned within lumen (1196). With slots (1103) in an expanded state, slots (1103) are configured to allow sleeves (1186, 1187) to deform outwardly from lumen (1196) with obturator (1092) contained within cannula (1194). Although cannula (1194) is depicted in association with obturator (1092), it should be understood that cannula (1194) may be utilized with obturator (92) described above or any other suitable obturator as will be apparent to those of ordinary skill in the art in view of the teachings herein.

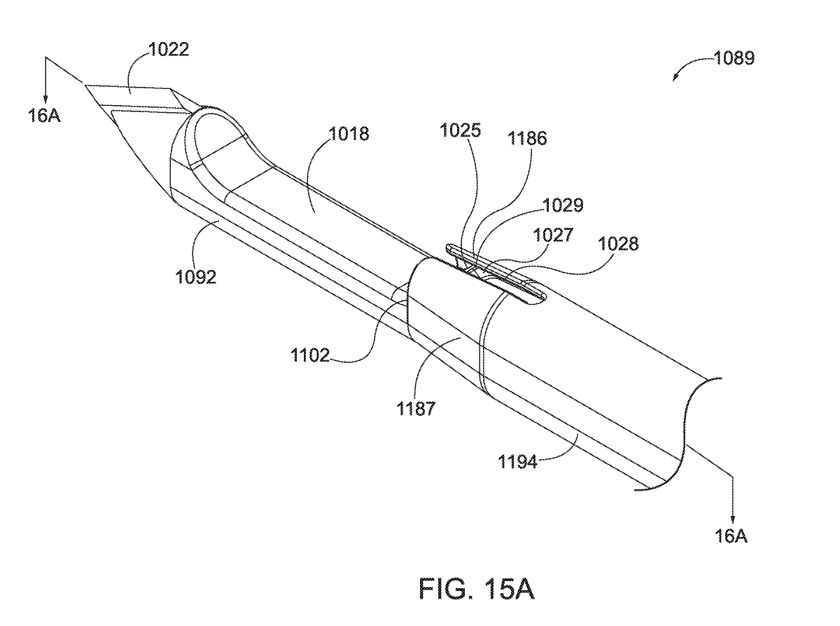

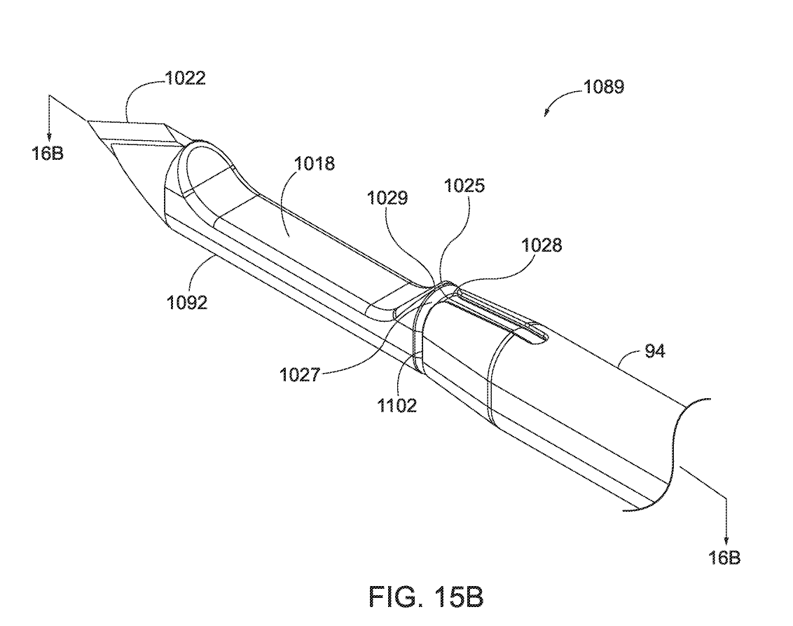

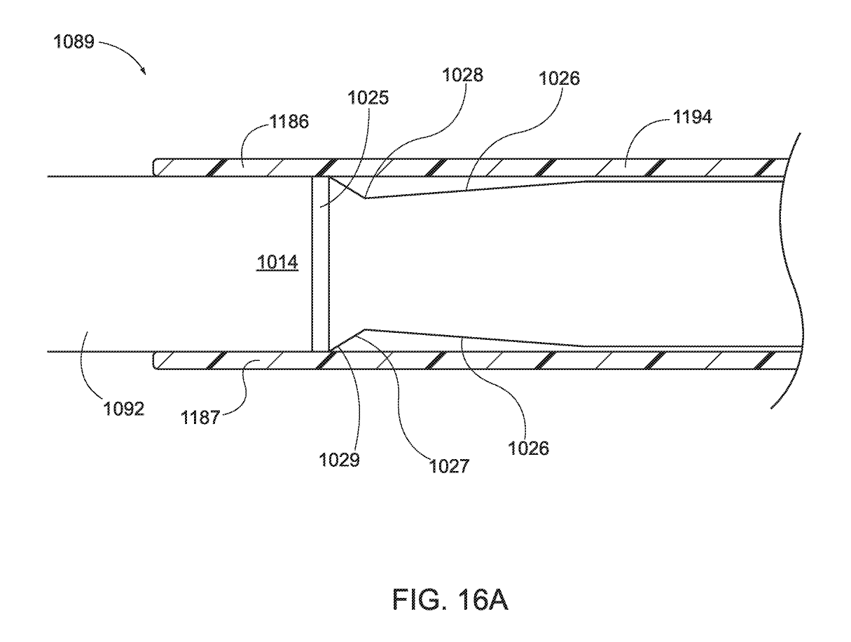

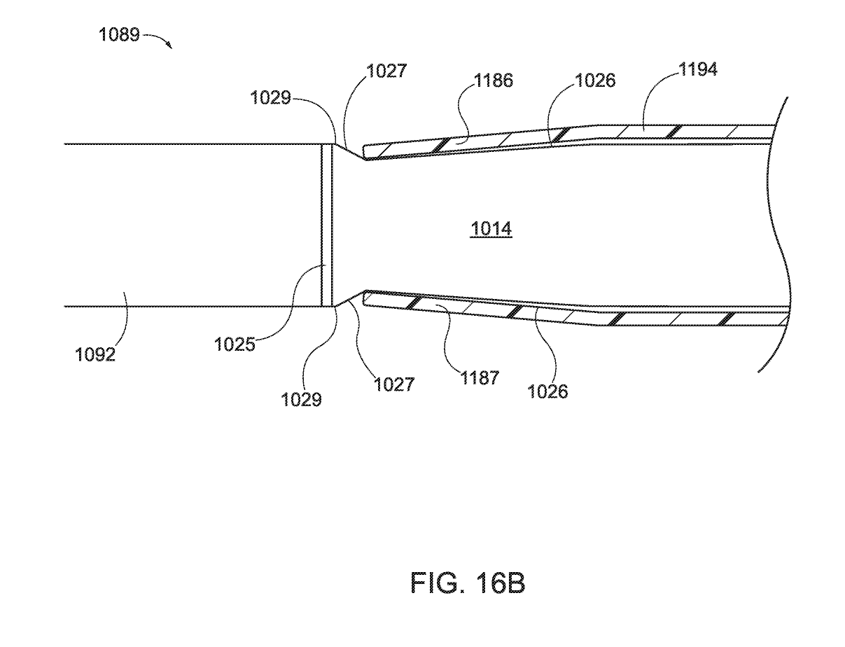

An exemplary insertion of obturator (1092) into cannula (1194) is shown in FIGS. 15A-16B. As best seen in FIGS. 15A and 16A, obturator (1092) is initially inserted into recess (1106) of cylindrical hub (1198) and received within lumen (1196) of cannula (1194) until piercing tip (1022) extends through open distal end (1102). At this point, open distal end (1102) extends beyond neck (1025) and thickness (1095) of cannula (1194) is not covered by the lateral extension of neck (1025) from shaft (1014).

As obturator (1092) is further inserted into cannula (1194), sleeves (1186, 1187) are resiliently deflected outwardly relative to lumen (1196) of cannula (1194) due to the force exerted onto sleeves (1186, 1187) by the lateral extension of neck (1025) from shaft (1014). As sleeves (1186, 1187) deform outwardly, slots (1103) transition to the expanded state to provide for the flexible deflection of sleeves (1186, 1187) which thereby accommodates obturator (1092) contained therein.

As obturator (1092) translates distally within cannula (1194), neck (1025) continues to apply a lateral force onto cannula (1194) until neck (1025) extends beyond lumen (1196) and open distal end (1102) of cannula (1194). At this stage, sleeves (1186, 1187) at open distal end (1102) move from neck (1025) inwardly along ramp (1027) until reaching proximal end (1028) of ramp (1027). With distal end (1102) of cannula (1194) positioned behind neck (1025), neck (1025) no longer exerts a lateral force upon sleeves (1186, 1187) from within lumen (1196) such that slots (1103) return to an unexpanded state and sleeves (1186, 1187) deform inwardly to engage tapered portion (1026), as best seen in FIGS. 15A and 16B. Thickness (1095) of cannula (1194) is fully covered by the lateral extension of neck (1025) from shaft (1014) due to the position of open distal end (1102) behind neck (1025) and sleeves (1186, 1187) engaging tapered portion (1026). In this instance, a substantially level surface is created along the external surface where open distal end (1102) is positioned along obturator (1092). After insertion of obturator (1092) into cannula (1194) is complete, an operator may insert the combination of obturator (1092) and cannula (1194) into a patient's body and minimize the occurrence of open distal end (1102) catching against the patient's skin or tissue and thus use less force to penetrate the patient's skin or tissue to reach the target biopsy site.

Although not shown, sleeves (1186, 1187) may further include a tapered thickness along a portion of cannula (1194) proximal to open distal end (1102) such that the cross-sectional area of cannula (1194) is further reduced. In this instance, sleeves (1186, 1187) are substantially covered behind neck (1025) of obturator (1092) when open distal end (1102) of cannula (1194) is positioned proximally relative to neck (1025).

To remove obturator (1092) from cannula (1194), obturator (1092) is proximally withdrawn from cannula (1194) thereby causing sleeves (1186, 1187) to engage proximal end (1028) of ramp (1027). Sleeves (1186, 1187) then begin to deform outwardly as sleeves (1186, 1187) approach distal end (1029) of ramp (1027). Once neck (1025) is retracted proximally relative to sleeves (1186, 1187), slots (1103) are transitioned to the expanded state and sleeves (1186, 1187) are laterally deflected away from lumen (1196). With open distal end (1102) positioned distally relative to neck (1025), and sleeves (1186, 1187) deflected outwardly from shaft (1014), obturator (1092) may next be removed from cannula (1194) entirely.

### VI. Exemplary Alternative Cannulas

A. Horizontal Split Sleeves

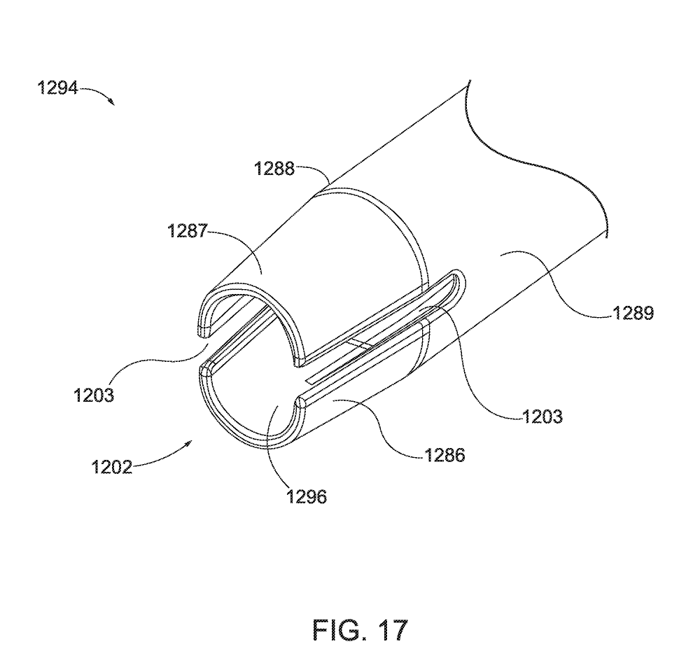

FIG. 17 shows an exemplary alternative cannula (1294) for use in association with probe (91) as similarly described above with respect to targeting set (89). Cannula (1294) is proximally attached to a cylindrical hub (not shown) and includes a lumen (1296) and slots (1203) proximal to an open distal end (1202). Similar to cannula (1194), the cylindrical hub includes a recess (not shown) that is in communication with lumen (1296) and is configured to receive obturator (1094) therein such that piercing tip (1022) extends distally from open distal end (1202) of cannula (1294). Slots (1203) extend proximally along cannula (1294) from open distal end (1202) and are positioned along a left wall (1289) and a right wall (1288) of cannula (1294) such that a top sleeve (1287) and a bottom sleeve (1286) are formed by slots (1203). Slots (1203) are sized and shaped to form an elliptical or oval opening along walls (1288, 1289) to thereby allow sleeves (1286, 1287) to flexibly deflect inwards or outwards relative to lumen (1296) by expanding or contracting slots (1203). Although slots (1203) are depicted as oval openings in FIG. 17, it should be understood that slots (1203) may be sized and shaped to form various suitable openings along the distal end portion of cannula (1294). Although two slots (1203) are depicted, it should be understood that more or less slots (1203) may be included along cannula (1294) as will be apparent to those of ordinary skill in the art.

Similar to sleeves (1186, 1187), sleeves (1286, 1287) extend along cannula (1294) at an inward angle towards open distal end (1202) such that sleeves (1286, 1287) are configured to deflect into lumen (1296). With obturator (1092) slidably received within lumen (1296) of cannula (1294), sleeves (1286, 1287) are configured to deform outwardly from lumen (1296) as neck (1025) of obturator (1092) has a diameter greater than the diameter of lumen (1296). Accordingly, slots (1203) are configured to expand due to the outward force applied onto sleeves (1286, 1287) by neck (1025) being positioned within lumen (1296). In this instance, with slots (1203) in an expanded state, slots (1203) are configured to permit sleeves (1286, 1287) to deform outwardly from lumen (1296) with neck (1025) slidably contained within cannula (1294). Although cannula (1294) is depicted in association with obturator (1092), it should be understood that cannula (1294) may be utilized with obturator (92) described above or any other suitable obturator as will be apparent to those of ordinary skill in the art in view of the teachings herein.

In the present example, obturator (1092) is slidably inserted into the recess of the cylindrical hub and received within lumen (1296) until piercing tip (1022) extends through open distal end (1202). In this instance, sleeves (1286, 1287) are resiliently deflected away from lumen (1296) due to the force exerted onto sleeves (1286, 1287) by the extension of neck (1025) from shaft (1014). As sleeves (1286, 1287) deform outwardly, slots (1203) transition to the expanded state to provide for the flexible deflection of sleeves (1286, 1287) which thereby accommodates obturator (1092) contained therein. As obturator (1092) translates distally within cannula (1294), neck (1025) ultimately extends beyond lumen (1296) and open distal end (1202) of cannula (1194). With neck (1025) no longer exerting a lateral force upon sleeves (1286, 1287) from within lumen (1296), slots (1203) return to an unexpanded state wherein sleeves (1286, 1287) deform inwardly and reassume an angled profile into lumen (1296).

Sleeves (1286, 1287) next deflect into lumen (1296) until encountering shaft (1014) of obturator (1092). Open distal end (1202) of cannula (1294) is positioned and received at proximal end (1028) of ramp (1027). With open distal end (1202) received behind neck (1025) and sleeves (1286, 1287) deflected inwardly towards shaft (1014), the combination of obturator (1092) and cannula (1294) forms a smaller cross-sectional area such that less resistance will be encountered from the insertion of obturator (1092) and cannula (1294) into a patient's body. With the reduction in cross-sectional area, an operator may use less force to penetrate the patient's skin or tissue to reach the target biopsy site. Although not shown, sleeves (1286, 1287) may further include a tapered thickness along a portion of cannula (1294) proximal to open distal end (1202) such that the cross-sectional area of cannula (1294) is further reduced. In this instance, sleeves (1286, 1287) are substantially covered behind neck (1025) of obturator (1092) when open distal end (1202) of cannula (1294) is positioned proximally relative to neck (1025).

To remove obturator (1092) from cannula (1294), obturator (1092) is proximally withdrawn from cannula (1294) thereby causing sleeves (1286, 1287) to engage proximal end (1028) of ramp (1027) and deform outwardly as sleeves (1286, 1287) approach distal end (1029) of ramp (1027). Once neck (1025) is retracted proximally relative to sleeves (1286, 1287), slots (1203) are transitioned to the expanded state and sleeves (1286, 1287) are laterally deflected away from lumen (1296). With open distal end (1202) positioned distally relative to neck (1025), and sleeves (1286, 1287) deflected outwardly from shaft (1014). Obturator (1092) may then be removed completely from cannula (1294).

B. Irregular Split Sleeves

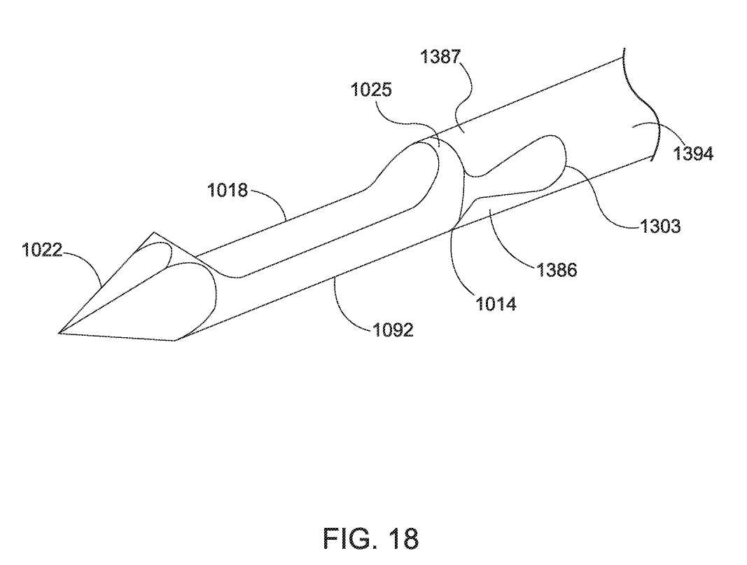

FIG. 18 shows another exemplary alternative cannula (1394) for use in association with probe (91) as similarly described above with respect to targeting set (89). Cannula (1394) is proximally attached to a cylindrical hub (not shown) and includes a lumen (1396) and slots (1303) proximal to an open distal end (1302). Similar to cannula (1194), the cylindrical hub includes a recess (not shown) that is in communication with lumen (1396) and is configured to receive obturator (1094) therein such that piercing tip (1022) extends distally from open distal end (1302) of cannula (1394). Slots (1303) extend proximally along cannula (1394) from open distal end (1302) and are positioned along a left wall (1389) and a right wall (1388) of cannula (1394) such that a top sleeve (1387) and a bottom sleeve (1386) are formed between slots (1303). Slots (1303) are sized and shaped to form an oblong, tear drop, or irregularly shaped opening along walls (1388, 1389) to thereby allow sleeves (1386, 1387) to flexibly deflect inwards or outwards relative to lumen (1396) by expanding or contracting slots (1303). Although irregularly shaped slots (1303) are depicted in FIG. 18, it should be understood that slots (1303) may be sized and shaped to form various suitable openings along the distal end portion of cannula (1394). Although two slots (1303) are depicted, it should be understood that more or less slots (1303) may be included along cannula (1394) as will be apparent to those of ordinary skill in the art.

Similar to sleeves (1186, 1187), sleeves (1386, 1387) extend along cannula (1394) at an inward angle towards open distal end (1302) such that sleeves (1386, 1387) are configured to deflect into lumen (1396). With obturator (1092) slidably received within lumen (1396) of cannula (1394), sleeves (1386, 1387) are configured to deform outwardly from lumen (1396) as neck (1025) of obturator (1092) has a diameter greater than the diameter of lumen (1396). Accordingly, slots (1303) are configured to expand due to the outward force applied onto sleeves (1386, 1387) by neck (1025) being positioned within lumen (1396). In this instance, with slots (1303) in an expanded state, slots (1303) are configured to allow sleeves (1386, 1387) to deform outwardly from lumen (1396) with neck (1025) slidably contained within cannula (1394). Although cannula (1394) is depicted in association with obturator (1092), it should be understood that cannula (1394) may be utilized with obturator (92) described above or any other suitable obturator as will be apparent to those of ordinary skill in the art in view of the teachings herein.

In the present example, obturator (1092) is slidably inserted into the recess of the cylindrical hub and received within lumen (1396) until piercing tip (1022) extends through open distal end (1302). In this instance, sleeves (1386, 1387) are resiliently deflected away from lumen (1396) due to the force exerted onto sleeves (1386, 1387) by the extension of neck (1025) from shaft (1014). As sleeves (1386, 1387) deform outwardly, slots (1303) transition to the expanded state to provide for the flexible deflection of sleeves (1386, 1387) which thereby accommodates obturator (1092) contained therein. As obturator (1092) translates distally within cannula (1394), neck (1025) ultimately extends beyond lumen (1396) and open distal end (1302) of cannula (1394). With neck (1025) no longer exerting a lateral force upon sleeves (1386, 1387) from within lumen (1396), slots (1303) return to an unexpanded state wherein sleeves (1386, 1387) deform inwardly and reassume an angled profile into lumen (1396).

Sleeves (1386, 1387) next deflect into lumen (1396) until encountering shaft (1014) of obturator (1092). Open distal end (1302) of cannula (1394) is positioned and received at proximal end (1028) of ramp (1027). With open distal end (1302) received behind neck (1025) and sleeves (1386, 1387) deflected inwardly towards shaft (1014), the combination of obturator (1092) and cannula (1394) forms a smaller cross-sectional area such that less resistance will be encountered from the insertion of obturator (1092) and cannula (1394) into a patient's body. With the reduction in cross-sectional area, an operator may use less force to penetrate the patient's skin or tissue to reach the target biopsy site. Although not shown, sleeves (1386, 1387) may further include a tapered thickness along a portion of cannula (1394) proximal to open distal end (1302) such that the cross-sectional area of cannula (1394) is further reduced. In this instance, sleeves (1386, 1387) are substantially covered behind neck (1025) of obturator (1092) when open distal end (1302) of cannula (1394) is positioned proximally relative to neck (1025).

To remove obturator (1092) from cannula (1394), obturator (1092) is proximally withdrawn from cannula (1394) thereby causing sleeves (1386, 1387) to engage proximal end (1028) of ramp (1027) and deform outwardly as sleeves (1386, 1387) approach distal end (1029) of ramp (1027). Once neck (1025) is retracted proximally relative to sleeves (1386, 1387), slots (1303) are transitioned to the expanded state and sleeves (1386, 1387) are laterally deflected away from lumen (1396). With open distal end (1302) positioned distally relative to neck (1025), and sleeves (1386, 1387) deflected outwardly from shaft (1014). Obturator (1092) may next be removed from cannula (1394) entirely.

C. Longitudinal Slit Sleeve

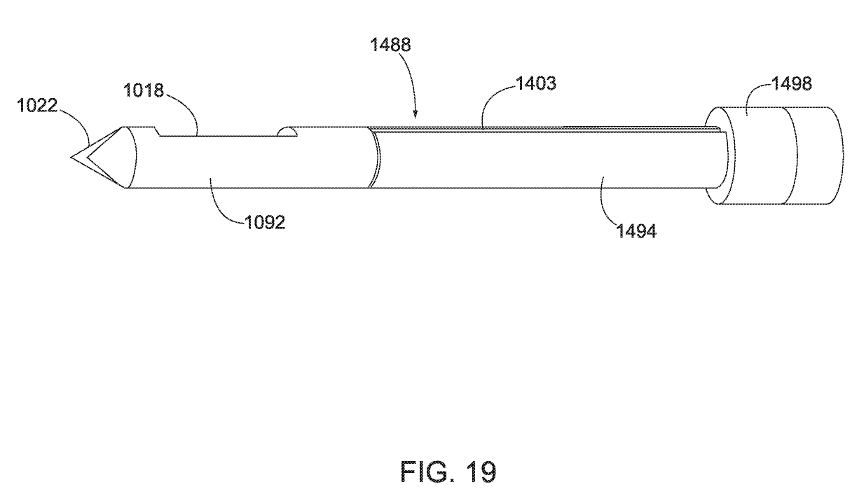

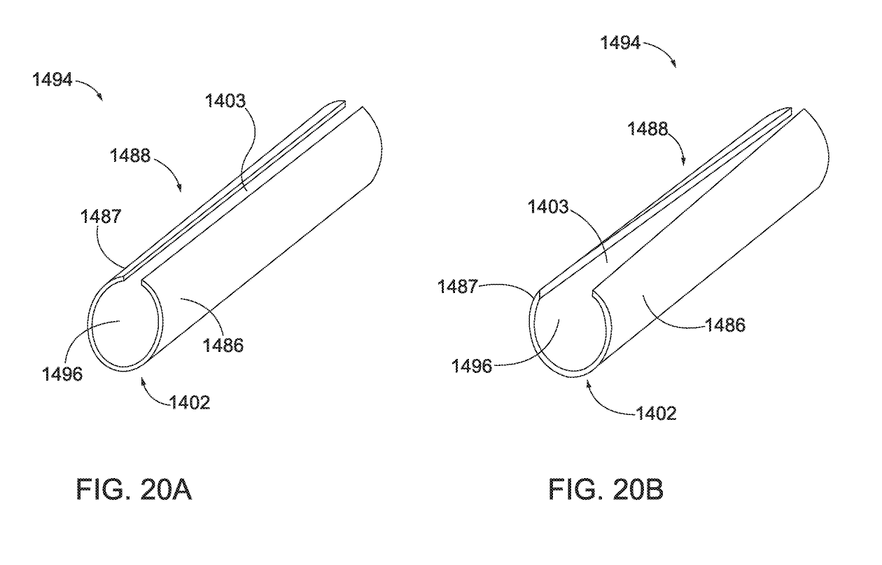

FIG. 19 shows another exemplary alternative cannula (1494) for use in association with probe (91) as similarly described above with respect to targeting set (89). Cannula (1494) is proximally attached to a cylindrical hub (1498) and includes a lumen (1496) and a longitudinal slit (1403) extending between an open distal end (1402) and cylindrical hub (1498). Similar to cannula (1194), cylindrical hub (1498) includes a recess (1406) that is in communication with lumen (1496) and is configured to receive obturator (1094) therein such that piercing tip (1022) extends distally from open distal end (1402) of cannula (1494). As seen in FIG. 20A, longitudinal slit (1403) extends along the longitudinal length of cannula (1494) and is positioned along top wall (1488) such that a left sleeve (1487) and a right sleeve (1486) are formed between longitudinal slit (1403). Longitudinal slit (1403) is sized and shaped to form an elliptical or rectangular opening along top wall (1488) to thereby allow sleeves (1486, 1487) to flexibly deflect away from each other by expanding longitudinal slit (1403). Although an elliptical longitudinal slit (1403) is depicted in FIG. 20A, it should be understood that longitudinal slit (1403) may be sized and shaped to form various suitable openings along the longitudinal length of cannula (1494). Although one longitudinal slit (1403) is depicted, it should be understood that more longitudinal slits (1403) may be included along cannula (1494) as will be apparent to those of ordinary skill in the art.

As further seen in FIG. 20B, sleeves (1486, 1487) are configured to deflect outwardly thereby expanding longitudinal slit (1403) along top wall (1488). With obturator (1092) slidably received within lumen (1496) of cannula (1494), sleeves (1486, 1487) are configured to deform laterally as neck (1025) of obturator (1092) has a diameter greater than the diameter of lumen (1496). Accordingly, longitudinal slit (1403) is configured to expand due to the outward force applied onto sleeves (1486, 1487) by neck (1025) being positioned within lumen (1496). In this instance, with longitudinal slit (1403) in an expanded state, longitudinal slit (1403) is configured to allow for sleeves (1486, 1487) to deform outwardly relative to top wall (1488) with neck (1025) slidably contained within cannula (1494). Although cannula (1494) is depicted in association with obturator (1092), it should be understood that cannula (1494) may be utilized with obturator (92) described above or any other suitable obturator as will be apparent to those of ordinary skill in the art in view of the teachings herein.

In the present example, obturator (1092) is slidably inserted into recess (1406) of cylindrical hub (1498) and received within lumen (1496) until piercing tip (1022) extends through open distal end (1402). In this instance, sleeves (1486, 1487) are resiliently deflected outward relative to top wall (1488) due to the force exerted onto sleeves (1486, 1487) by the extension of neck (1025) from shaft (1014). As sleeves (1486, 1487) deform laterally, longitudinal slit (1403) transitions to the expanded state to provide for the flexible deflection of sleeves (1486, 1487) which thereby accommodates obturator (1092) contained therein, as seen in FIG. 20B. As obturator (1092) translates distally within cannula (1494), neck (1025) ultimately advances beyond open distal end (1402) and out of lumen (1496). With neck (1025) no longer exerting a lateral force upon sleeves (1486, 1487) from within lumen (1496), longitudinal slit (1403) returns to an unexpanded state wherein sleeves (1486, 1487) deform inwardly and reassume a parallel alignment with each other along top wall (1488), as seen in FIG. 20A.

In this instance, sleeves (1486, 1487) are separated from each other by longitudinal slit (1403), with the opening of longitudinal slit (1403) being uniform on top wall (1488) along the longitudinal length of cannula (1494). Open distal end (1402) of cannula (1494) is positioned and received at proximal end (1028) of ramp (1027), as seen in FIG. 19. With open distal end (1402) received behind neck (1025) and sleeves (1486, 1487) not laterally deflected along top wall (1488), the combination of obturator (1092) and cannula (1494) forms a smaller cross-sectional area such that less resistance will be encountered from the insertion of obturator (1092) and cannula (1494) into a patient's body. With the reduction in cross-sectional area, an operator may use less force to penetrate the patient's skin or tissue to reach the target biopsy site. Although not shown, sleeves (1486, 1487) may further include a tapered thickness along a portion of cannula (1494) proximal to open distal end (1402) such that the cross-sectional area of cannula (1494) is further reduced. In this instance, sleeves (1486, 1487) are substantially covered behind neck (1025) of obturator (1092) when open distal end (1402) of cannula (1494) is positioned proximally relative to neck (1025).

To remove obturator (1092) from cannula (1494), obturator (1092) is proximally withdrawn from cannula (1494) thereby causing sleeves (1486, 1487) to engage proximal end (1028) of ramp (1027) and deform outwardly as sleeves (1486, 1487) approach distal end (1029) of ramp (1027). Once neck (1025) is retracted proximally relative to sleeves (1486, 1487), longitudinal slit (1403) is transitioned to the expanded state and sleeves (1486, 1487) are laterally deflected relative to top wall (1488), as seen in FIG. 20B. With open distal end (1402) positioned distally relative to neck (1025), and sleeves (1486, 1487) deflected outwardly from shaft (1014), the combination of obturator (1092) and cannula (1494) forms a larger cross-sectional area. Obturator (1092) may next be removed from cannula (1494) entirely.

### VII. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A targeting set for use with positioning a biopsy device within a patient, the targeting set comprising: an obturator including an elongate shaft, a tissue receiving feature, and a neck portion; wherein the elongate shaft defines an internal lumen; wherein the neck portion is configured to extend laterally from the elongate shaft such that the neck portion protrudes around a perimeter of the elongate shaft.

### Example 2

The targeting set of Example 1, wherein the neck portion is positioned along the elongate shaft proximally relative to the tissue receiving feature.

### Example 3

The targeting set of Example 1, wherein the obturator is insertable into a lumen of an introducer cannula in lieu of a needle of a biopsy device.

### Example 4

The targeting set of Example 1, wherein the obturator further includes a sharp distal tip, wherein the tissue receiving feature is positioned proximally of the sharp distal tip.

### Example 5

The targeting set of Example 1, further including a cannula.

### Example 6

The targeting set of Example 5, wherein the cannula comprises a shaft defining a lumen extending longitudinally through the shaft.

### Example 7

The targeting set of Example 6, wherein the lumen of the cannula is configured to receive the obturator.

### Example 8

The targeting set of Example 7, wherein the cannula further comprises a lateral aperture, wherein the lateral aperture of the cannula is configured to align with the tissue receiving feature of the obturator when the obturator is received within the lumen of the cannula.

### Example 9

The targeting set of any one or more of Examples 7 through 8, wherein the cannula further comprises an open distal end, wherein the obturator is configured to extend distally past the open distal end of the cannula when the obturator is received within the lumen of the cannula.

### Example 10

A targeting set for use with positioning a biopsy device within a patient, the targeting set comprising: a cannula including a shaft extending between an open distal end and an open proximal end, a pair of slots proximal to the open distal end such that the pair of slots define a pair of sleeves; wherein the pair of sleeves are configured to deform outwardly from the shaft such that the pair of slots are operable to expand.

### Example 11

The targeting set of Example 10, wherein the pair of sleeves are angled inwardly relative to the shaft such that the pair of sleeves are configured to deflect towards each other.

### Example 12

The targeting set of Example 11, wherein the shaft is defined by a top wall, bottom wall, left wall and right wall.

### Example 13

The targeting set of Example 12, wherein the pair of slots are positioned along the left wall and the right wall such that the pair of sleeves are positioned along the top wall and the bottom wall.

### Example 14

The targeting set of Example 12, wherein the pair of slots are positioned along the top wall and the left wall such that the pair of sleeves are positioned along the left wall and the right wall.

### Example 15

The targeting set of Example 13 through Example 14, wherein the pair of slots have an elliptical or oval shape.

### Example 16

The targeting set of Example 13 through Example 14, wherein the pair of slots have an oblong or irregular shape.

### Example 17

The targeting set of Example 11, wherein the shaft defines a lumen extending longitudinally through the shaft, wherein the lumen is configured to receive an obturator.

### Example 18

The targeting set of Example 17, wherein the pair of sleeves are configured to deform outwardly relative to the shaft with the obturator slidably received within the lumen.

### Example 19

The targeting set of Example 18, wherein the obturator includes an elongate shaft, and a tissue receiving feature; wherein the elongate shaft defines an internal lumen.

### Example 20

The targeting set of Example 19, wherein the cannula further comprises a lateral aperture, wherein the lateral aperture of the cannula is configured to align with the tissue receiving feature of the obturator when the obturator is received within the lumen of the cannula.

### Example 21

The targeting set of Example 20, wherein the obturator is configured to extend distally past the open distal end of the cannula when the obturator is received within the lumen of the cannula.

### Example 22

The targeting set of Example 19, wherein the obturator further includes a neck portion configured to extend laterally from the elongate shaft such that the neck portion protrudes around a perimeter of the elongate shaft.

### Example 23

The targeting set of Example 19 through Example 22, wherein the neck portion is configured to deform the pair of sleeves outwardly relative to the shaft when the obturator is received within the lumen of the cannula.

### Example 24

The targeting set of Example 19 through Example 23, wherein the pair of sleeves are configured to deflect inwardly towards the elongate shaft when the neck portion is distally extended beyond the open distal end of the cannula.

### Example 25

A targeting set for use with positioning a biopsy device within a patient, the targeting set comprising: a cannula including a shaft extending between an open distal end and an open proximal end, a longitudinal slit extending along the shaft and between the open distal end and the open proximal end; wherein the longitudinal slit defines a pair of sleeves; wherein the pair of sleeves are configured to deform outwardly from the shaft such that the longitudinal slit is operable to expand.

### Example 26

The targeting set of Example 25, wherein the shaft is defined by a top wall, bottom wall, left wall and right wall.

### Example 27

The targeting set of Example 26, wherein the longitudinal slit is positioned along the top wall such that the pair of sleeves are positioned along the left wall and the right wall.

### Example 28

The targeting set of Example 27, wherein the shaft defines a lumen extending longitudinally through the shaft, wherein the lumen is configured to receive an obturator.

### Example 29

The targeting set of Example 28, wherein the pair of sleeves are configured to deform outwardly relative to the shaft with the obturator slidably received within the lumen.

### Example 30

The targeting set of Example 29, wherein the obturator includes an elongate shaft, and a tissue receiving feature; wherein the elongate shaft defines an internal lumen;

### Example 31

The targeting set of Example 30, wherein the obturator is configured to extend distally past the open distal end of the cannula when the obturator is received within the lumen of the cannula.

### Example 32

The targeting set of Example 31, wherein the obturator further includes a neck portion configured to extend laterally from the elongate shaft such that the neck portion protrudes around a perimeter of the elongate shaft.

### Example 33

The targeting set of Example 32, wherein the neck portion is configured to deform the pair of sleeves outwardly relative to the shaft when the obturator is received within the lumen of the cannula.

### Example 34

The targeting set of Example 33, wherein the longitudinal slit is operable to expand when the pair of sleeves deform outwardly.

### Example 35

The targeting set of Example 32, wherein the pair of sleeves are configured to substantially cover the obturator when the neck portion is distally extended beyond the open distal end of the cannula.

### Example 36

A targeting set for use with positioning a biopsy device relative to a patient, the targeting set comprising: an obturator including a sharp tip and a tapered portion oriented proximally of the sharp tip; and a cannula including a shaft extending between an open distal end and an open proximal end, a pair of slots proximal to the open distal end such that the pair of slots define a pair of sleeves; wherein the pair of sleeves are configured to deform relative to the shaft such that the pair of slots are operable to expand around a portion of the obturator and into the tapered portion.

### Example 37

The targeting set of Example 36, wherein the pair of sleeves are angled inwardly relative to the shaft such that the pair of sleeves are configured to deflect towards each other.

### Example 38

The targeting set of any one or more of Examples 36 and 37, wherein the shaft is defined by a top wall, bottom wall, left wall and right wall, wherein the pair of slots are positioned along the left wall and the right wall such that the pair of sleeves are positioned along the top wall and the bottom wall.

### Example 39

The targeting set of any one or more of Examples 36 and 37, wherein the shaft is defined by a top wall, bottom wall, left wall and right wall, wherein the pair of slots are positioned along the top wall and the left wall such that the pair of sleeves are positioned along the left wall and the right wall.

### Example 40

A targeting set for use with positioning a biopsy device within a patient, the targeting set comprising: an obturator defining an indented portion; and a cannula including a shaft extending between an open distal end and an open proximal end, a longitudinal slit extending along the shaft and between the open distal end and the open proximal end; wherein the longitudinal slit defines a pair of sleeves; wherein the pair of sleeves are configured to deform outwardly from the shaft such that the longitudinal slit is operable to expand, wherein the pair of sleeves are further configured to deform inwardly for receipt within the indented portion of the obturator.

### Example 41

The targeting set of Example 40, wherein the obturator further includes a sharp distal tip, wherein the indented portion is positioned proximally of the sharp distal tip.

### Example 42

The targeting set of any one or more of Examples 40 and 41, wherein the shaft of the cannula defines a lumen extending between the open distal end and the open proximal end, wherein the lumen is configured to receive the obturator, wherein the cannula is configured to receive the obturator such that the obturator extends distally past the open distal end of the cannula.

### Example 43

The targeting set of any one or more of Examples 40 through 42, wherein the obturator further includes a neck portion adjacent to the indented portion, wherein the pair of sleeves are configured to deform outwardly from the shaft of the cannula to accommodate the neck of the obturator.

### Example 44

The targeting set of any one or more of Examples 40 through 43, wherein the cannula and the obturator are configured to define a substantially smooth surface when the pair of sleeves are received within the indented portion of the obturator.

### V. Conclusion

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Embodiments of the present invention have application in conventional endoscopic and open surgical instrumentation as well as application in robotic-assisted surgery.

By way of example only, embodiments described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a medical facility. A device may also be sterilized using any other technique known in the art, including but not limited to beta or gamma radiation, ethylene oxide, or steam.

Embodiments of the devices disclosed herein can be reconditioned for reuse after at least one use. Reconditioning may include any combination of the steps of disassembly of the device, followed by cleaning or replacement of particular pieces, and subsequent reassembly. In particular, embodiments of the devices disclosed herein may be disassembled, and any number of the particular pieces or parts of the devices may be selectively replaced or removed in any combination. Upon cleaning and/or replacement of particular parts, embodiments of the devices may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A targeting set for use with positioning a biopsy needle relative to a patient, the targeting set comprising:
an obturator including an elongate shaft extending between a distal tip and a hub;
a side notch defined by the elongate shaft and configured to receive tissue for visualization under MRI; and
a neck defined by the elongate shaft and disposed proximate of the side notch, wherein the neck has a tapered portion tapering radially inwardly towards the distal tip.

2. The targeting set of claim 1, wherein the neck includes an outwardly extending portion, wherein the outwardly extending portion extends radially outwardly from the tapered portion.

3. The targeting set of claim 1, further comprising a cannula having a cannula hub and adapted to receive the obturator, the cannula hub having a lock adapted to lock the obturator hub, wherein the neck includes an outwardly extending portion extending radially outwardly from the tapered portion so as to provide a smooth transition between the proximal end of the side notch and the distal end of the cannula when the cannula hub and sleeve hub are locked.

4. The targeting set of claim 1, wherein the neck includes an outwardly extending portion distally of the tapered portion such that the neck tapers radially inwardly through the tapered portion and then extends radially outwardly from the tapered portion towards the tip.

5. The targeting set of claim 1, further comprising a cannula configured to receive the obturator, wherein the cannula includes a distal end configured for receipt within the neck of the obturator.

6. The targeting set of claim 1, further comprising a cannula configured to receive the obturator, wherein the cannula includes a distal end defining at least one sleeve having a slot, wherein the at least one sleeve is configured to flex for receipt of the sleeve within the neck of the obturator.

7. The targeting set of claim 1, further comprising a cannula configured to receive the obturator, wherein the cannula includes a distal end defining a pair of sleeves separated by a pair of slots, wherein the pair of sleeves are configured to flex for receipt of the pair of sleeves within the neck of the obturator.

8. The targeting set of claim 1, further comprising a cannula configured to receive the obturator, wherein the cannula and the obturator each define an oval-shaped cross-section such that the oval-shaped cross-section defines a longitudinal axis and a transverse axis oriented perpendicularly relative to the longitudinal axis, wherein the cannula defines a distal end having a pair of sleeves separated by a pair of slots, wherein the pair of slots are aligned with the longitudinal axis defined by the oval-shaped cross-section.

9. The targeting set of claim 1, further comprising a cannula configured to receive the obturator, wherein the neck further includes a ramp extending outwardly from the tapered portion, wherein the cannula includes a distal end configured for receipt within the neck of the obturator, wherein the distal end of the cannula is configured to expand upon engagement with the ramp when the cannula is advanced distally relative to the obturator.

10. The targeting set of claim 1, further comprising a cannula configured to receive the obturator, wherein the cannula includes a distal end defining a pair of sleeves separated by a pair of slots, wherein the pair of sleeves are configured to flex for receipt within the neck of the obturator, wherein each slot of the pair of slots defines a tear drop-shaped slot.

11. The targeting set of claim 1, further comprising a cannula configured to receive the obturator, wherein the cannula includes a distal end configured for receipt within the neck of the obturator, wherein the cannula is configured to define a smooth transition between the obturator and the cannula when the distal end of the cannula is received within the neck of the obturator.

12. The targeting set of claim 1, further comprising a cannula configured to receive the obturator, wherein the neck includes a tapered portion and an outwardly extending portion, wherein the tapered portion tapers inwardly to an inner diameter relative to the outer diameter of the elongate shaft as the tapered portion extends distally towards the outwardly extending portion, wherein the cannula has a thickness approximately equivalent to one half of the difference between the inner diameter and the outer diameter.

13. The targeting set of claim 1, wherein the obturator defines a lumen extending through the shaft, wherein the lumen is in communication with the side notch.

14. The targeting set of claim 1, wherein the neck is disposed proximally relative to the side notch.

15. The targeting set of claim 1, further comprising a cannula configured to receive the obturator, wherein the cannula defines an oval-shape including a pair of flat sides and a lumen extending longitudinally therethrough, wherein the lumen of the cannula is configured to receive the obturator, wherein the cannula further includes an open distal end and a relief portion adjacent to the open distal end, wherein the open distal end of the cannula is configured to nest with the neck portion of the obturator when the obturator is received within the cannula, wherein the relief portion of the cannula includes one or more slits, wherein the one or more slits are aligned with the pair of flat sides and define a tear drop shape.

16. A targeting set for use with positioning a biopsy device relative to a patient, the targeting set comprising:
an obturator including a sharp distal tip adapted to pierce tissue, a side notch positioned proximally of the sharp distal tip and configured to receive tissue for visualization under MRI, and a tapered portion positioned proximally of the side notch; and
a cannula extending between an open distal end and a proximal end, the cannula including a pair of sleeves adjacent to the open distal end wherein the pair of sleeves are separated by a pair of slots;
wherein the pair of sleeves are configured to deform such that when the obturator is inserted into the cannula, the pair of sleeves are operable to expand around the side notch and then contract proximally of the side notch to provide a smooth transition between the proximal end of the side notch and the distal end of the pair of sleeves.

17. The targeting set of claim 16, wherein the pair of sleeves are angled inwardly relative to the cannula.

18. The targeting set of claim 16, wherein the cannula is defined by a top wall, bottom wall, left wall and right wall, wherein the pair of slots are positioned along the left wall and the right wall and the pair of sleeves are positioned along the top wall and the bottom wall.

19. The targeting set of claim 16, wherein the cannula is defined by a top wall, bottom wall, left wall and right wall, wherein the pair of slots are positioned along the top wall and the bottom wall and the pair of sleeves are positioned along the left wall and the right wall.

20. A targeting set for use with positioning a biopsy device within a patient, the targeting set comprising:
an obturator defining an indented portion; and
a cannula including a shaft extending between an open distal end and a proximal end, the cannula further including a longitudinal slit extending along the shaft between the open distal end and the proximal end;
wherein the longitudinal slit is defined by a sleeve; wherein the sleeve is configured to deform outwardly from the obturator such that the longitudinal slit is operable to expand, wherein the sleeve is further configured to deform inwardly for receipt within the indented portion of the obturator.
